(19) United States
(12) Patent Application Publication
(10) Pub. No.: US 2025/0264476 A1
(43) Pub. Date: Aug. 21, 2025

(54) AMINO ACID-SPECIFIC BINDER AND SELECTIVELY IDENTIFYING AN AMINO ACID

(71) Applicant: Government of the United States of America, as represented by the Secretary of Commerce, Gaithersburg, MD (US)

(72) Inventors: Zvi Kelman, Gaithersburg, MD (US); John Paul Marino, Silver Spring, MD (US); Nicholas William Callahan, Gaithersburg, MD (US)

(21) Appl. No.: 18/559,515

(22) PCT Filed: May 9, 2022
(86) PCT No.: PCT/US2022/028400

§ 371 Date: Nov. 7, 2023

Related U.S. Application Data
(60) Provisional application No. 63/185,483, filed on May 7, 2021.

(51) Int. Cl. G01N 33/68; C07K 14/195; G01N 33/58

(52) U.S. Cl. CPC G01N 33/6842; C07K 14/195; G01N 33/582; G01N 33/6824

(57) ABSTRACT

N-terminal amino acid binding (NAAB) reagents are a tool for parallel, high-throughput proteomics. A fluorescently-labeled NAAB allows immobilized peptides to be identified by their N-terminal residues using single-molecule fluorescent microscopy, which enables novel proteomic analysis, like fluorescence-based next-generation protein sequence. Amino acid-specific binders are presented which bind to leucine at the N-terminus of peptides/polypeptides/proteins with high affinity and specificity, allowing detection of an N-terminal leucine with high confidence.

### CROSS REFERENCE TO RELATED APPLICATIONS

This application claims the benefit of U.S. Provisional Patent Application Ser. No. 63/185,483 (filed May 7, 2021), which is herein incorporated by reference in its entirety.

### STATEMENT REGARDING FEDERALLY SPONSORED RESEARCH

This invention was made with United Stated Government support from the National Institute of Standards and Technology (NIST), an agency of the United Stated Department of Commerce. The Government has certain rights to this invention.

### BRIEF DESCRIPTION

Disclosed is an amino acid-specific binder for selectively binding to an amino acid in an analyte, the amino acid-specific binder comprising:
a first amino acid sequence comprising

(Sequence ID No. 1)
ASVVPQEGQQVTRKHYPNYKVIVLNDDFNTFQHVVACLKKYIPNMISDRA
WELTNQVHYGGRAIVWVGPQEQAELYHEQLLRAGLTMAPLEPE;

a second amino acid sequence comprising

(Sequence ID No. 2)
ASVVPQERQQDTRKHYPNYKVIVLNDDFNTFRHVAACLMKYIPNMTSDRA
WELTNQVHYEGQAIVWVGPQEQAELYHEQLLRAGLTMAPLEPE;

a third amino acid sequence comprising

(Sequence ID No. 3)
ASVVPQERQQVARKHYPNYKVIVLNDDFNTFRHVAACLMKYIPNMTSDRA
WELTNQVHYEGQAIVWVGPQVQAELYHEQLLRAGLTMAPLEPG;

a fourth amino acid sequence comprising

(Sequence ID No. 4)
ASVVPQERQQDTRKHYPNYKVIVLNDDENTFRHVAACLMKYIPNKTSDRA
WELTNQVHYEGQAIVWVGPQEQAELYHEQLLRAGLTMAPLEPE;

a fifth amino acid sequence comprising

(Sequence ID No. 5)
ASVVPQERQQVTRKHYPNYKVIVLNDDFNTFRHVAACLMKYIPNMTSDRA
WELTNQVHYKGQAIVWVGPQEQAELNHEQLLRAGLTMAPLEPE;

a sixth amino acid sequence comprising

(Sequence ID No. 6)
ASVVPQERQQVARKHYPNYKVIVLNDDFNTFRHVAACLMKYIPNMTSDR
AWELTNQVHYEGQAIVWVGPQEQAELYHEQLLRAGLTMAPLEPE;

a seventh amino acid sequence comprising

(Sequence ID No. 7)
ASVVPQERQQDTRKHYPNYKVIVLNDDFNTFRHVAACLMKYIPNMTSDR
AWELTNQVHYKGQAIVWVGPQVQAELYHEQLLRAGLTMAPLEPG;

an eighth amino acid sequence comprising

(Sequence ID No. 8)
ASVVPQERQQDTRKHYPNYKVIVLNDDFNTFRHVAACLMKYIPNMTSDR
AWELTNQVHYEGQAIVWVGPQVQAELYHEQLLRAGLTMAPLEPG;

a ninth amino acid sequence comprising

(Sequence ID No. 9)
ASVVPQEGQQVTRKHYPNYKVIVLNDDFNTFQHVVACLKKYIPNMISDR
AWELTNQVHYGGRAIVWVGPQEQAELNHEQLLRAGLTMAPLEPE;

a tenth amino acid sequence comprising, or

(Sequence ID No. 10)
ASVVPQERQQVARKHYPNYKVIVLNDDFNTFRHVAACLMKYIPNMTSDR
AWELTNQVHYEGQAIVWVGPQVQAELNHEQLLRAGLTMAPLEPE.

Disclosed is an amino acid-specific binder for selectively binding to an amino acid in an analyte, the amino acid-specific binder comprising an amino acid sequence with a homology of at least 30% compared to an amino acid sequence comprising:
a first amino acid sequence comprising

(Sequence ID No. 1)
ASVVPQEGQQVTRKHYPNYKVIVLNDDFNTFQHVVACLKKYIPNMISDR
AWELTNQVHYGGRAIVWVGPQEQAELYHEQLLRAGLTMAPLEPE;

a second amino acid sequence comprising

(Sequence ID No. 2)
ASVVPQERQQDTRKHYPNYKVIVLNDDFNTFRHVAACLMKYIPNMTSDR
AWELTNQVHYEGQAIVWVGPQEQAELYHEQLLRAGLTMAPLEPE;

a third amino acid sequence comprising

(Sequence ID No. 3)
ASVVPQERQQVARKHYPNYKVIVLNDDFNTFRHVAACLMKYIPNMTSDR
AWELTNQVHYEGQAIVWVGPQVQAELYHEQLLRAGLTMAPLEPG;

a fourth amino acid sequence comprising

(Sequence ID No. 4)
ASVVPQERQQDTRKHYPNYKVIVLNDDFNTFRHVAACLMKYIPNKTSDR
AWELTNQVHYEGQAIVWVGPQEQAELYHEQLLRAGLTMAPLEPE;

a fifth amino acid sequence comprising

(Sequence ID No. 5)
ASVVPQERQQVTRKHYPNYKVIVLNDDFNTFRHVAACLMKYIPNMTSDR
AWELTNQVHYKGQAIVWVGPQEQAELNHEQLLRAGLTMAPLEPE;

a sixth amino acid sequence comprising

(Sequence ID No. 6)
ASVVPQERQQVARKHYPNYKVIVLNDDFNTFRHVAACLMKYIPNMTSDR
AWELTNQVHYEGQAIVWVGPQEQAELYHEQLLRAGLTMAPLEPE;

a seventh amino acid sequence comprising

(Sequence ID No. 7)
ASVVPQERQQDTRKHYPNYKVIVLNDDFNTFRHVAACLMKYIPNMTSDR
AWELTNQVHYKGQAIVWVGPQVQAELYHEQLLRAGLTMAPLEPG;

an eighth amino acid sequence comprising

(Sequence ID No. 8)
ASVVPQERQQDTRKHYPNYKVIVLNDDFNTFRHVAACLMKYIPNMTSDR
AWELTNQVHYEGQAIVWVGPQVQAELYHEQLLRAGLTMAPLEPG;

a ninth amino acid sequence comprising

(Sequence ID No. 9)
ASVVPQEGQQVTRKHYPNYKVIVLNDDFNTFQHVVACLKKYIPNMISDR
AWELTNQVHYGGRAIVWVGPQEQAELNHEQLLRAGLTMAPLEPE;

a tenth amino acid sequence comprising, or

(Sequence ID No. 10)
ASVVPQERQQVARKHYPNYKVIVLNDDFNTFRHVAACLMKYIPNMTSDR
AWELTNQVHYEGQAIVWVGPQVQAELNHEQLLRAGLTMAPLEPE.

Disclosed is a binder complex for selectively identifying an amino acid, the binder complex comprising: an amino acid-specific binder, and an adjunct attached to the amino acid-specific binder, wherein the amino acid-specific binder binds selectively to a binding amino acid, and the amino acid-specific binder comprises:
a first amino acid sequence comprising

(Sequence ID No. 1)
ASVVPQEGQQVTRKHYPNYKVIVLNDDFNTFQHVVACLKKYIPNMISDR
AWELTNQVHYGGRAIVWVGPQEQAELYHEQLLRAGLTMAPLEPE;

a second amino acid sequence comprising

(Sequence ID No. 2)
ASVVPQERQQDTRKHYPNYKVIVLNDDFNTFRHVAACLMKYIPNMTSDR
AWELTNQVHYEGQAIVWVGPQEQAELYHEQLLRAGLTMAPLEPE;

a third amino acid sequence comprising

(Sequence ID No. 3)
ASVVPQERQQVARKHYPNYKVIVLNDDFNTFRHVAACLMKYIPNMTSDR
AWELTNQVHYEGQAIVWVGPQVQAELYHEQLLRAGLTMAPLEPG;

a fourth amino acid sequence comprising

(Sequence ID No. 4)
ASVVPQERQQDTRKHYPNYKVIVLNDDFNTFRHVAACLMKYIPNKTSDR
AWELTNQVHYEGQAIVWVGPQEQAELYHEQLLRAGLTMAPLEPE;

a fifth amino acid sequence comprising

(Sequence ID No. 5)
ASVVPQERQQVTRKHYPNYKVIVLNDDFNTFRHVAACLMKYIPNMTSDR
AWELTNQVHYKGQAIVWVGPQEQAELNHEQLLRAGLTMAPLEPE;

a sixth amino acid sequence comprising

(Sequence ID No. 6)
ASVVPQERQQVARKHYPNYKVIVLNDDFNTFRHVAACLMKYIPNMTSDR
AWELTNQVHYEGQAIVWVGPQEQAELYHEQLLRAGLTMAPLEPE;

a seventh amino acid sequence comprising

(Sequence ID No. 7)
ASVVPQERQQDTRKHYPNYKVIVLNDDFNTFRHVAACLMKYIPNMTSDR
AWELTNQVHYKGQAIVWVGPQVQAELYHEQLLRAGLTMAPLEPG;

an eighth amino acid sequence comprising

(Sequence ID No. 8)
ASVVPQERQQDTRKHYPNYKVIVLNDDFNTFRHVAACLMKYIPNMTSDR
AWELTNQVHYEGQAIVWVGPQVQAELYHEQLLRAGLTMAPLEPG;

a ninth amino acid sequence comprising

(Sequence ID No. 9)
ASVVPQEGQQVTRKHYPNYKVIVLNDDFNTFQHVVACLKKYIPNMISDR
AWELTNQVHYGGRAIVWVGPQEQAELNHEQLLRAGLTMAPLEPE;

a tenth amino acid sequence comprising, or

(Sequence ID No. 10)
ASVVPQERQQVARKHYPNYKVIVLNDDFNTFRHVAACLMKYIPNMTSDR
AWELTNQVHYEGQAIVWVGPQVQAELNHEQLLRAGLTMAPLEPE;

or an eleventh amino acid sequence with a homology of at least 30% compared to an amino acid sequence comprising the first amino acid sequence, the second amino acid sequence, the third amino acid sequence, the fourth amino acid sequence, the fifth amino acid sequence, the sixth amino acid sequence, the seventh amino acid sequence, the eighth amino acid sequence, the ninth amino acid sequence, or the tenth amino acid sequence.

Disclosed is a process for selectively identifying an N-terminal amino acid, the process comprising: providing an analyte; contacting a C-terminal end of the analyte with an anchor; anchoring the C-terminal end to the anchor to form an anchored analyte; contacting an N-terminal amino acid of the anchored analyte with a binder complex, the binder complex comprising: an amino acid-specific binder; and a taggant attached to the amino acid-specific binder; selectively binding the amino acid-specific binder of the binder complex to the N-terminal amino acid of the anchored analyte when the N-terminal amino acid is a binding amino acid to form a tagged complex; subjecting the taggant of the tagged complex to a stimulus; producing, by the taggant of the tagged complex, a taggant signal in response to the stimulus; detecting the taggant signal; and identifying the N-terminal amino acid based on the taggant signal, wherein the amino acid-specific binder binds selectively to the binding amino acid, and the amino acid-specific binder comprises:
a first amino acid sequence comprising

(Sequence ID No. 1)
ASVVPQEGQQVTRKHYPNYKVIVLNDDFNTFQHVVACLKKYIPNMISDR
AWELTNQVHYGGRAIVWVGPQEQAELYHEQLLRAGLTMAPLEPE;

a second amino acid sequence comprising

(Sequence ID No. 2)
ASVVPQERQQDTRKHYPNYKVIVLNDDFNTFRHVAACLMKYIPNMTSDR
AWELTNQVHYEGQAIVWVGPQEQAELYHEQLLRAGLTMAPLEPE;

a third amino acid sequence comprising

(Sequence ID No. 3)
ASVVPQERQQVARKHYPNYKVIVLNDDFNTFRHVAACLMKYIPNMTSDR
AWELTNQVHYEGQAIVWVGPQVQAELYHEQLLRAGLTMAPLEPG;

a fourth amino acid sequence comprising

(Sequence ID No. 4)
ASVVPQERQQDTRKHYPNYKVIVLNDDFNTFRHVAACLMKYIPNKTSDR
AWELTNQVHYEGQAIVWVGPQEQAELYHEQLLRAGLTMAPLEPE;

a fifth amino acid sequence comprising

(Sequence ID No. 5)
ASVVPQERQQVTRKHYPNYKVIVLNDDFNTFRHVAACLMKYIPNMTSDR
AWELTNQVHYKGQAIVWVGPQEQAELNHEQLLRAGLTMAPLEPE;

a sixth amino acid sequence comprising

(Sequence ID No. 6)
ASVVPQERQQVARKHYPNYKVIVLNDDFNTFRHVAACLMKYIPNMTSDR
AWELTNQVHYEGQAIVWVGPQEQAELYHEQLLRAGLTMAPLEPE;

a seventh amino acid sequence comprising

(Sequence ID No. 7)
ASVVPQERQQDTRKHYPNYKVIVLNDDFNTFRHVAACLMKYIPNMTSDR
AWELTNQVHYKGQAIVWVGPQVQAELYHEQLLRAGLTMAPLEPG;

an eighth amino acid sequence comprising

(Sequence ID No. 8)
ASVVPQERQQDTRKHYPNYKVIVLNDDFNTFRHVAACLMKYIPNMTSDR
AWELTNQVHYEGQAIVWVGPQVQAELYHEQLLRAGLTMAPLEPG;

a ninth amino acid sequence comprising

(Sequence ID No. 9)
ASVVPQEGQQVTRKHYPNYKVIVLNDDFNTFQHVVACLKKYIPNMISDR
AWELTNQVHYGGRAIVWVGPQEQAELNHEQLLRAGLTMAPLEPE;

a tenth amino acid sequence comprising, or

(Sequence ID No. 10)
ASVVPQERQQVARKHYPNYKVIVLNDDFNTFRHVAACLMKYIPNMTSDR
AWELTNQVHYEGQAIVWVGPQVQAELNHEQLLRAGLTMAPLEPE;

or an eleventh amino acid sequence with a homology of at least 30% compared to an amino acid sequence comprising the first amino acid sequence, the second amino acid sequence, the third amino acid sequence, the fourth amino acid sequence, the fifth amino acid sequence, the sixth amino acid sequence, the seventh amino acid sequence, the eighth amino acid sequence, the ninth amino acid sequence, or the tenth amino acid sequence.

Disclosed is a process for selectively isolating an analyte, the process comprising: contacting an amino acid-specific binder with an analyte comprising a protein, a peptide, an amino acid, or a combination comprising at least one of foregoing; selectively binding the amino acid-specific binder to the N-terminal amino acid of the analyte when the N-terminal amino acid is a binding amino acid to form an isolation complex; separating the isolation complex from a fluid in which the isolation complex is disposed to selectively isolating the analyte, wherein the amino acid-specific binder binds selectively to the binding amino acid, and the amino acid-specific binder comprises:
a first amino acid sequence comprising

(Sequence ID No. 1)
ASVVPQEGQQVTRKHYPNYKVIVLNDDFNTFQHVVACLKKYIPNMISDR
AWELTNQVHYGGRAIVWVGPQEQAELYHEQLLRAGLTMAPLEPE;

a second amino acid sequence comprising

(Sequence ID No. 2)
ASVVPQERQQDTRKHYPNYKVIVLNDDFNTFRHVAACLMKYIPNMTSDR
AWELTNQVHYEGQAIVWVGPQEQAELYHEQLLRAGLTMAPLEPE;

a third amino acid sequence comprising

(Sequence ID No. 3)
ASVVPQERQQVARKHYPNYKVIVLNDDFNTFRHVAACLMKYIPNMTSDR
AWELTNQVHYEGQAIVWVGPQVQAELYHEQLLRAGLTMAPLEPG;

a fourth amino acid sequence comprising

(Sequence ID No. 4)
ASVVPQERQQDTRKHYPNYKVIVLNDDFNTFRHVAACLMKYIPNKTSDR
AWELTNQVHYEGQAIVWVGPQEQAELYHEQLLRAGLTMAPLEPE;

a fifth amino acid sequence comprising

(Sequence ID No. 5)
ASVVPQERQQVTRKHYPNYKVIVLNDDFNTFRHVAACLMKYIPNMTSDR
AWELTNQVHYKGQAIVWVGPQEQAELNHEQLLRAGLTMAPLEPE;

a sixth amino acid sequence comprising

(Sequence ID No. 6)
ASVVPQERQQVARKHYPNYKVIVLNDDFNTFRHVAACLMKYIPNMTSDR
AWELTNQVHYEGQAIVWVGPQEQAELYHEQLLRAGLTMAPLEPE;

a seventh amino acid sequence comprising

(Sequence ID No. 7)
ASVVPQERQQDTRKHYPNYKVIVLNDDFNTFRHVAACLMKYIPNMTSDR
AWELTNQVHYKGQAIVWVGPQVQAELYHEQLLRAGLTMAPLEPG;

an eighth amino acid sequence comprising

(Sequence ID No. 8)
ASVVPQERQQDTRKHYPNYKVIVLNDDFNTFRHVAACLMKYIPNMTSDR
AWELTNQVHYEGQAIVWVGPQVQAELYHEQLLRAGLTMAPLEPG;

a ninth amino acid sequence comprising

(Sequence ID No. 9)
ASVVPQEGQQVTRKHYPNYKVIVLNDDFNTFQHVVACLKKYIPNMISDR
AWELTNQVHYGGRAIVWVGPQEQAELNHEQLLRAGLTMAPLEPE;

a tenth amino acid sequence comprising, or

(Sequence ID No. 10)
ASVVPQERQQVARKHYPNYKVIVLNDDFNTFRHVAACLMKYIPNMTSDR
AWELTNQVHYEGQAIVWVGPQVQAELNHEQLLRAGLTMAPLEPE;

or an eleventh amino acid sequence with a homology of at least 30% compared to an amino acid sequence comprising the first amino acid sequence, the second amino acid sequence, the third amino acid sequence, the fourth amino acid sequence, the fifth amino acid sequence, the sixth amino acid sequence, the seventh amino acid sequence, the eighth amino acid sequence, the ninth amino acid sequence, or the tenth amino acid sequence.

### BRIEF DESCRIPTION OF THE DRAWINGS

The following description should not be considered limiting in any way. With reference to the accompanying drawings, like elements are numbered alike.

FIG. 1 shows an amino acid-specific binder selectively bound to an amino acid that is a binding amino acid of an analyte in panel A. and panel B shows a non-binding amino acid unbound to an amino acid-specific binder;

FIG. 2 shows an amino acid-specific binder of a binder complex selectively bound to a binding amino acid of an analyte in panel A, and panel B shows a non-binding amino acid unbound to an amino acid-specific binder of a binder complex;

FIG. 3 shows a binder complex in an absence of an intervening member in panel A and inclusion of an intervening member in panel B;

FIG. 4 shows a binder complex that includes an amino acid-specific binder attached to a taggant in panel A, a protein in panel B, a chemical modifier in panel C, and a substrate in panel D;

FIG. 5 shows a binder complex that includes a plurality of amino acid-specific binders attached to an adjunct in panel A and panel B and attached to a plurality of adjuncts in panel C;

FIG. 6 shows an analyte that includes a binding amino acid in panel A, a non-binding amino acid in panel B, a binding amino acid and non-binding amino acid in panel C. and an analyte that is a peptide or protein that includes a plurality of amino acids in panel D and panel E;

FIG. 7 shows an anchored analyte in an absence of an intervening member in panel A and inclusion of an intervening member in panel B;

FIG. 8 shows an anchored analyte in panel A; and a tagged complex in panel B;

FIG. 9 shows an anchored analyte in panel A; in panel B, the anchored analyte shown in panel A after removal of an N-terminal amino acid; in panel C, the anchored analyte shown in panel B after removal of an N-terminal amino acid; and in panel D, a tagged complex with production of a taggant signal;

FIG. 10 shows an anchored analyte in panel A; in panel B, the anchored analyte shown in panel A after production of an inert residue from an N-terminal amino acid; and in panel C, the anchored analyte shown in panel B after production of another inert residue;

FIG. 11 shows, in panel A, an isolated complex that includes an amino acid-specific binder selectively bound to an analyte; in panel B, an isolated complex that includes an amino acid-specific binder selectively bound an N-terminal amino acid that is a binding amino acid in an analyte; and in panel C, an isolated complex that includes an amino acid-specific binder of a binder complex selectively bound to an N-terminal amino acid that is a binding amino acid in an analyte;

FIG. 12 shows formation of a tagged complex and detection of a taggant signal;

FIG. 13 shows formation of a tagged complex and detection of a taggant signal;

FIG. 14 shows a fluorescent labeling for detection of peptide binding during flow cytometry, wherein myc tag 260 is detected with fluorescent label taggant 214 on anti-myc antibody 256. Peptide 248 is detected using streptavidin-PE 258 that binds biotin 264 attached to C-terminus 224 of peptide 248. It should be appreciated that there are other tags besides myc and different fluorophores;

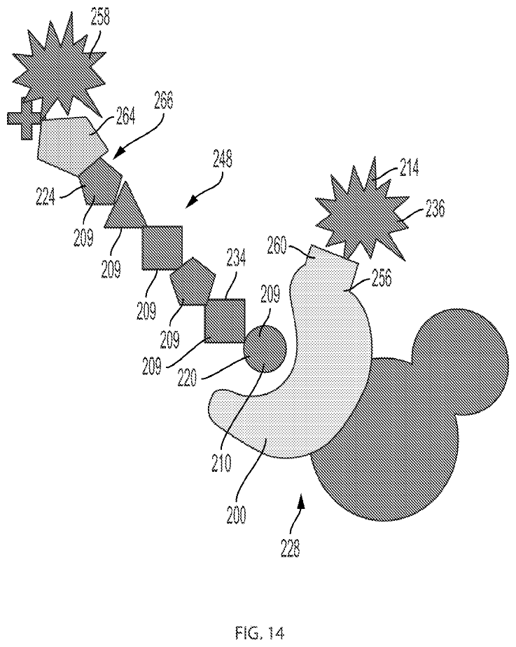

FIG. 15 shows an expected flow cytometry result for yeast that displays a non-binding protein in quadrant 1 (Q1), yeast that binds the peptide in Q2, yeast that does not display the protein in Q3, and yeast that exhibits non-specific binding to the peptide in Q4;

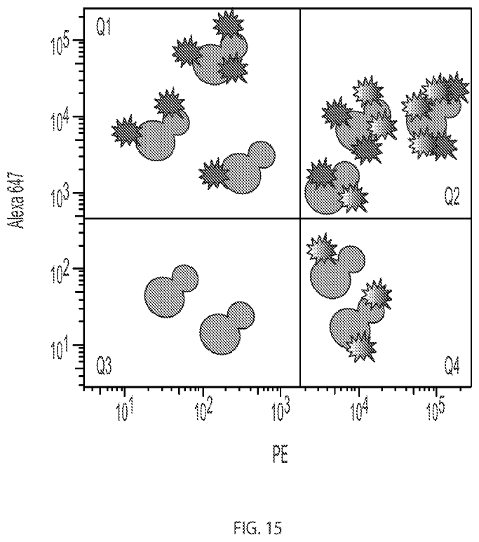

FIG. 16 shows a graph of fluorescent taggant fluorescence versus phycoerythrin (PE) fluorescence for flow cytometry plots displaying PE fluorescence seen in a naïve library against a leucine (Leu) peptide, wherein Q2 correspond to cells carried on to a next round of sorting after outgrowth;

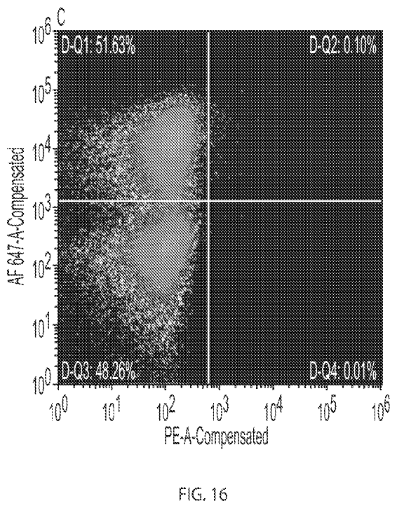

FIG. 17 shows a graph of fluorescent taggant fluorescence versus PE fluorescence for flow cytometry plots displaying increased PE fluorescence seen in each round after the data shown in FIG. 15 and an additional 3 rounds of selection of a library against a Leu peptide;

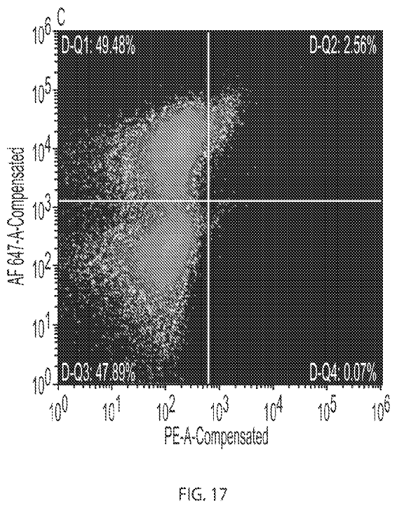

FIG. 18 shows the number of cells counted in Q2, as determined via fluorescent taggant fluorescence versus PE fluorescence for flow cytometry plots, showing the magnitude of difference in binding against Phe, Tyr, Trp, and a variety of Leu peptides between a naïve library and a library resulting from three rounds of selection;

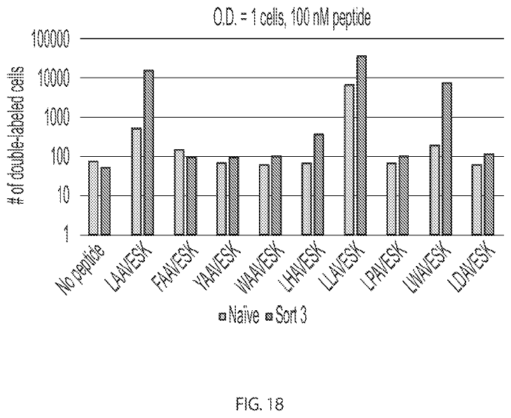

FIG. 19 shows alignment of Thermosynechococcus elongatus ClpS2 (Sequence ID No. 11), Agrobacterium tumefaciens ClpS2 (Sequence ID. No. 12), and an engineered NAAB-Phe protein sequences. T. elongatus ClpS2 has 29% sequence identity to A. tumefaciens ClpS2 and 31% sequence identity to the engineered NAAB-Phe. Positions of library-selected mutations for T. elongatus ClpS2 are indicated by pointers. Sequence features used to distinguish phylum of ClpS variant are boxed; and

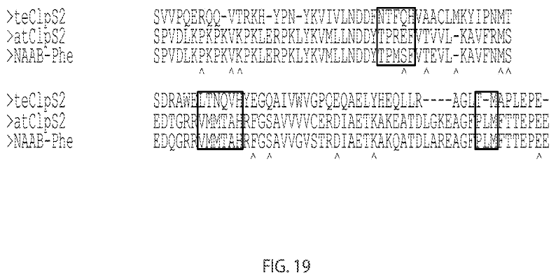

FIG. 20 shows the association curve of T. elongatus ClpS2 with the Leu peptide variant and the association curve with the same Leu peptide for a composite protein sequence which incorporates the mutants from the library generated from T. elongatus ClpS2.

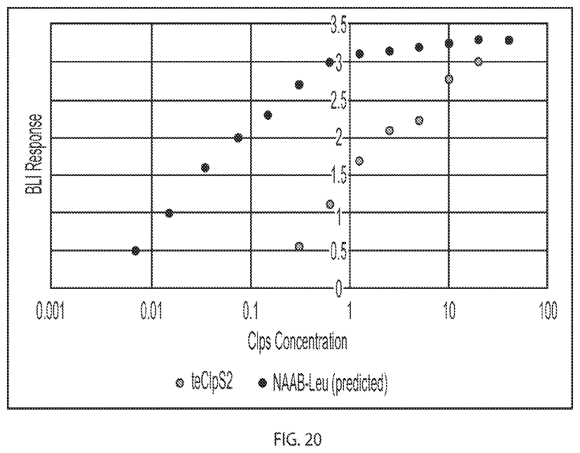

### DETAILED DESCRIPTION

A detailed description of one or more embodiments is presented herein by way of exemplification and not limitation.

It has been discovered that an amino acid-specific binder herein selectively binds to a binding amino acid selected from a group of specific amino acids. Indeed, the amino acid-specific binder overcomes a central challenge in single-molecule protein sequencing technology and provides high-fidelity, sequential recognition, detection of specific amino acids that can be included in a peptide sequence. Moreover, the amino acid-specific binder overcomes lack of selectivity involved with an N-End Rule Pathway adaptor protein (NERPap). ClpS, that natively recognizes an N-terminal amino acid (NAA) on a peptide chain, wherein the NERPap lacks selectivity and affinity for peptide sequencing Beneficially and unexpectedly, the amino acid-specific binder provides selectivity by including novel sequence variants of T. elongatus ClpS2, a ClpS protein, such that the amino acid-specific binder has enhanced affinity and selectivity for the amino acid leucine (Leu), which can occur as a single binding amino acid or at an N-terminus of a peptide or protein. Advantageously, the amino acid-specific binder determines a sequence or fingerprint of amino acids in a peptide or protein when used iteratively.

Amino acid-specific binder 200 selectively binds to binding amino acid 210 in analyte 212. In an embodiment, amino acid-specific binder 200 is a first amino acid sequence including X1-GQQVT-X2-QHVV-X3-K-X4-MI-X5-GGR-X6-E-X7-Y-X8-E; a second amino acid sequence including X1-RQQDT-X2-RHVA-X3-M-X4-MT-X5-EGQ-X6-E-X7-Y-X8-E; a third amino acid sequence including X1-RQQVA-X2-RHVA-X3-M-X4-MT-X5-EGQ-X6-V-X7-Y-X8-G; a fourth amino acid sequence including X1-RQQDT-X2-RHVA-X3-M-X4-KT-X5-EGQ-X6-E-X7-Y-X8-E; a fifth amino acid sequence including X1-RQQVT-X2-RHVA-X3-M-X4-MT-X5-KGQ-X6-E-X7-N-X8-E; a sixth amino acid sequence including X1-RQQVA-X2-RHVA-X3-M-X4-MT-X5-EGQ-X6-E-X7-Y-X8-E; a seventh amino acid sequence including X1-RQQDT-X2-RHVA-X3-M-X4-MT-X5-KGQ-X6-V-X7-Y-X8-G; or an eighth amino acid sequence including X1-RQQDT-X2-RHVA-X3-M-X4-MT-X5-EGQ-X6-V-X7-Y-X8-G, wherein. X1 includes an amino acid sequence including ASVVPQE (Sequence ID No. 20); X2 includes an amino acid sequence including RKHYPNYKVIVLNDDFNTF; X3 includes an amino acid sequence including ACL (Sequence ID No. 21); X4 includes an amino acid sequence including KYIPN (Sequence ID No. 22); X5 includes an amino acid sequence including SDRAWELTNQVHY (Sequence ID No. 23); X6 includes an amino acid sequence including AIVWVGPQ (Sequence ID No. 24); X7 includes an amino acid sequence including QAEL (Sequence ID No. 25); and X8 includes an amino acid sequence including HEQLLRAGLTMAPLEP (Sequence ID No. 26), such that a total percentage amount of substitutions and deletions to X1, X2. X3, X4, X5. X6, X7, and X8 is from 0% to less than 30%, exclusive of PSVVPQERQQVTRKHYPNYKVIVLNDDFNTFQHVAACLMKYIPNMTSDRAWELTN QVHYEGQAIVWVGPQEQAELYHEQLLRAGLTMAPLEPE (Sequence ID No. 27).

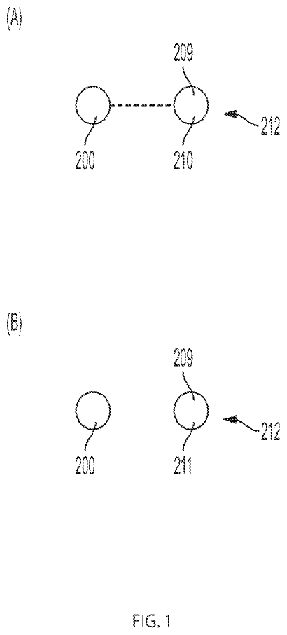

In an embodiment, amino acid-specific binder 200 is a protein that includes an amino acid that sequence is ASVVPQEGQQVTRKHYPNYKVIVLNDDFNTFQHVVACLKKYIPNMISDRAWELTNQ VHYGGRAIVWVGPQEQAELYHEQLLRAGLTMAPLEPE (Sequence ID No. 1); ASVVPQERQQDTRKHYPNYKVIVLNDDFNTFRHVAACLMKYIPNMTSDRAWELTN QVHYEGQAIVWVGPQEQAELYHEQLLRAGLTMAPLEPE (Sequence ID No. 2); ASVVPQERQQVARKHYPNYKVIVLNDDENTFRHVAACLMKYIPNMTSDRAWELTN QVHYEGQAIVWVGPQVQAELYHEQLLRAGLTMAPLEPG (Sequence ID No. 3); ASVVPQERQQDTRKHYPNYKVIVLNDDFNTFRHVAACLMKYIPNKTSDRAWELTN QVHYEGQAIVWVGPQEQAELYHEQLLRAGLTMAPLEPE (Sequence ID No. 4); ASVVPQERQQVTRKHYPNYKVIVLNDDENTFRHVAACLMKYIPNMTSDRAWELTN QVHYKGQAIVWVGPQEQAELNHEQLLRAGLTMAPLEPE (Sequence ID No. 5); ASVVPQERQQVARKHYPNYKVIVLNDDFNTFRHVAACLMKYIPNMTSDRAWELTN QVHYEGQAIVWVGPQEQAELYHEQLLRAGLTMAPLEPE (Sequence ID No. 6); ASVVPQERQQDTRKHYPNYKVIVLNDDFNTFRHVAACLMKYIPNMTSDRAWELTN QVHYKGQAIVWVGPQVQAELYHEQLLRAGLTMAPLEPG (Sequence ID No. 7); ASVVPQERQQDTRKHYPNYKVIVLNDDFNTFRHVAACLMKYIPNMTSDRAWELTN QVHYEGQAIVWVGPQVQAELYHEQLLRAGLTMAPLEPG (Sequence ID No. 8); ASVVPQEGQQVTRKHYPNYKVIVLNDDFNTFQHVVACLKKYIPNMISDRAWELTNQ VHYGGRAIVWVGPQEQAELNHEQLLRAGLTMAPLEPE (Sequence ID No. 9); ASVVPQERQQVARKHYPNYKVIVLNDDFNTFRHVAACLMKYIPNMTSDRAWELTN QVHYEGQAIVWVGPQVQAELNHEQLLRAGLTMAPLEPE (Sequence ID No. 10); or an amino acid sequence with a homology of at least 30% compared to an amino acid sequence comprising the amino acid sequence with Sequence ID No. 1, Sequence ID No. 2, Sequence ID No. 3, Sequence ID No. 4, Sequence ID No. 5, Sequence ID No. 6, Sequence ID No. 7, Sequence ID No. 8, Sequence ID No. 9, or Sequence ID No. 10. Amino acid-specific binder 200 binds selectively to binding amino acid 210 selected from the group consisting of isoleucine, leucine, phenylalanine, tryptophan, tyrosine, and valine; and chemically modified amino acids phenylalanine, tryptophan, tyrosine, isoleucine, leucine, and valine. Accordingly, with reference to FIG. 1, amino acid-specific binder 200 selectively binds to binding amino acid 210 of analyte 212 but does not bind to non-binding amino acid 211.

In an embodiment, the amino acid sequence includes:

(Sequence ID No. 1)
ASVVPQEGQQVTRKHYPNYKVIVLNDDFNTFQHVVACLKKYIPNMISDR
AWELTNQVHYGGRAIVWVGPQEQAELYHEQLLRAGLTMAPLEPE;
(Sequence ID No. 2)
ASVVPQERQQDTRKHYPNYKVIVLNDDENTFRHVAACLMKYIPNMTSDR
AWELTNQVHYEGQAIVWVGPQEQAELYHEQLLRAGLTMAPLEPE;
(Sequence ID No. 3)
ASVVPQERQQVARKHYPNYKVIVLNDDFNTFRHVAACLMKYIPNMTSDR
AWELTNQVHYEGQAIVWVGPQVQAELYHEQLLRAGLTMAPLEPG;
(Sequence ID No. 4)
ASVVPQERQQDTRKHYPNYKVIVLNDDFNTFRHVAACLMKYIPNKTSDR
AWELTNQVHYEGQAIVWVGPQEQAELYHEQLLRAGLTMAPLEPE;
(Sequence ID No. 5)
ASVVPQERQQVTRKHYPNYKVIVLNDDENTFRHVAACLMKYIPNMTSDR
AWELTNQVHYKGQAIVWVGPQEQAELNHEQLLRAGLTMAPLEPE;
(Sequence ID No. 6)
ASVVPQERQQVARKHYPNYKVIVLNDDFNTFRHVAACLMKYIPNMTSDR
AWELTNQVHYEGQAIVWVGPQEQAELYHEQLLRAGLTMAPLEPE;
(Sequence ID No. 7)
ASVVPQERQQDTRKHYPNYKVIVLNDDFNTFRHVAACLMKYIPNMTSDR
AWELTNQVHYKGQAIVWVGPQVQAELYHEQLLRAGLTMAPLEPG;
(Sequence ID No. 8)
ASVVPQERQQDTRKHYPNYKVIVLNDDFNTFRHVAACLMKYIPNMTSDR
AWELTNQVHYEGQAIVWVGPQVQAELYHEQLLRAGLTMAPLEPG;
(Sequence ID No. 9)
ASVVPQEGQQVTRKHYPNYKVIVLNDDFNTFQHVVACLKKYIPNMISDR
AWELTNQVHYGGRAIVWVGPQEQAELNHEQLLRAGLTMAPLEPE;
or
(Sequence ID No. 10)
ASVVPQERQQVARKHYPNYKVIVLNDDFNTFRHVAACLMKYIPNMTSDR
AWELTNQVHYEGQAIVWVGPQVQAELNHEQLLRAGLTMAPLEPE.

According to an embodiment, amino acid-specific binder 200 is a protein with Sequence ID No. 1 and binds selectively to leucine.

According to an embodiment, amino acid-specific binder 200 is a protein with Sequence ID No. 2 and binds selectively to leucine.

According to an embodiment, amino acid-specific binder 200 is a protein with Sequence ID No. 3 and binds selectively to leucine.

According to an embodiment, amino acid-specific binder 200 is a protein with Sequence ID No. 4 and binds selectively to leucine.

According to an embodiment, amino acid-specific binder 200 is a protein with Sequence ID No. 5 and binds selectively to leucine.

According to an embodiment, amino acid-specific binder 200 is a protein with Sequence ID No. 6 and binds selectively to leucine.

According to an embodiment, amino acid-specific binder 200 is a protein with Sequence ID No. 7 and binds selectively to leucine.

According to an embodiment, amino acid-specific binder 200 is a protein with Sequence ID No. 8 and binds selectively to leucine.

According to an embodiment, amino acid-specific binder 200 is a protein with Sequence ID No. 9 and binds selectively to leucine.

According to an embodiment, amino acid-specific binder 200 is a protein with Sequence ID No. 10 and binds selectively to leucine.

According to an embodiment, amino acid-specific binder 200 is a protein with a sequence homology of at least 30% compared to an amino acid sequence selected from the group consisting essentially of the amino acid sequence with Sequence ID No. 1, Sequence ID No. 2, Sequence ID No. 3. Sequence ID No. 4, Sequence ID No. 5, Sequence ID No. 6. Sequence ID No. 7, Sequence ID No. 8, Sequence ID No. 9, and Sequence ID No. 10 and binds selectively to leucine

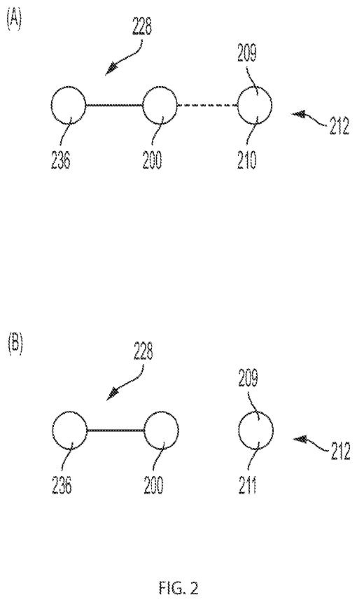

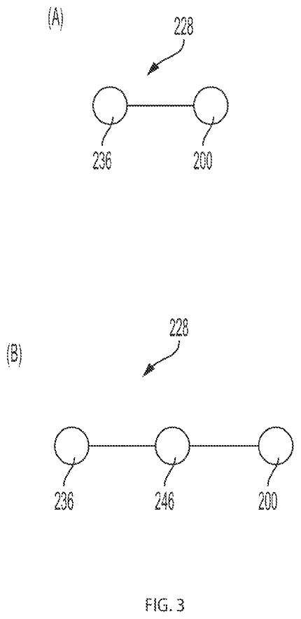

Binder complex 228 selectively identifies an amino acid. In an embodiment, binder complex 228 includes amino acid-specific binder 200 and adjunct 236 attached to amino acid-specific binder 200. Attachment of adjunct 236 to amino acid-specific binder 200 can include a covalent bond, an ionic bond, electrostatic interaction (e.g., a π-cation interaction, dipole-dipole interaction, a multi-pole interaction, and the like), intercalation, a clathrate arrangement (e.g., with adjunct 236 partially or wholly trapped in amino acid-specific binder 200 or vice-versa, such that amino acid-specific binder 200 can still selectively bind to binding amino acid 210, e.g., of analyte 212), and the like. Further, adjunct 236 can be attached to amino acid-specific binder 200 either directly, indirectly, or a combination thereof. With reference to FIG. 3, when adjunct 236 is directly attached to amino acid-specific binder 200, direct attachment occurs in an absence of an intervening member between adjunct 236 and amino acid-specific binder 200 as shown in panel A. When adjunct 236 is indirectly attached to amino acid-specific binder 200 as shown in panel B, indirect attachment occurs in a presence of the intervening member 246 between adjunct 236 and amino acid-specific binder 200. Accordingly, with reference to FIG. 2, amino acid-specific binder 200 selectively binds to binding amino acid 210 of analyte 212 but does not bind to non-binding amino acid 211.

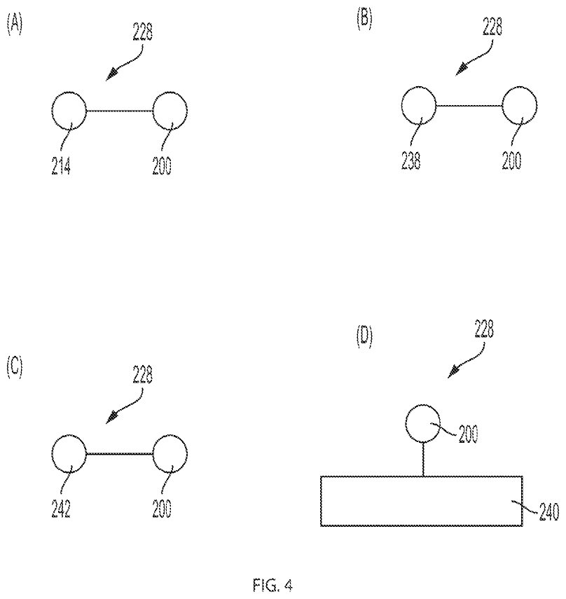

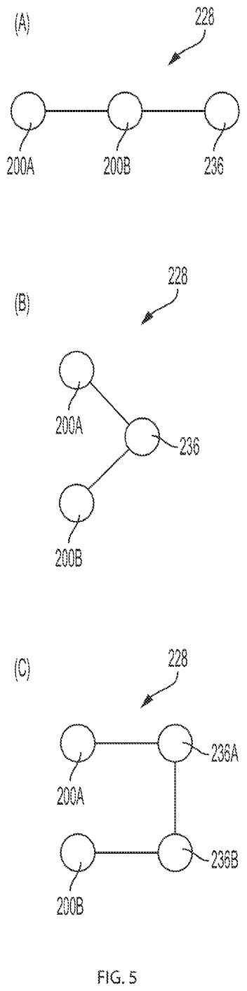

In binder complex 228, with reference to FIG. 3, adjunct 236 can determine a position or identity of amino acid-specific binder 200 and determine if amino acid-specific binder 200 is bound to analyte 212. Adjunct 236 can be taggant 214, protein 238, substrate 240, chemical modifier 242, or a combination thereof, e.g., as shown in FIG. 4. In an embodiment, adjunct 236 includes taggant 214. In an embodiment, adjunct 236 includes a substrate such that analyte 212 can be immobilized when in contact with amino acid-specific binder 200. Binder complex 228 can include an arbitrary number of amino acid-specific binder 200 and adjunct 236 that can be connectedly attached in an arbitrary arrangement as shown in FIG. 5.

Protein 238 can include a protein to facilitate expression or purification of amino-acid specific binder 200 such as a protein with a functional group that can be immobilized on a resin, an antibody, Protein A, Protein G, a peptide of six histidine residues. Glutathione S-transferase, maltose binding protein, biotin, or streptavidin. Moreover, protein 238 can include a protein with a reactive property such as enzymatic activity, a protease cleavage site, or fluorescence that can be stimulated to produce a signal and can be green fluorescent protein, horseradish peroxidase, luciferase, and the like. Moreover, protein 238 can include proteins with a selected molecular weight, isoelectric point, or functional group that can facilitate separation of binding complex 238, e.g., by dialysis, chromatography, or gradient centrifugation Exemplary proteins 238 include an immunoglobulin, a high molecular weight protein (HMWP), DNA-binding protein, oligosaccharide binding protein, and the like. In an embodiment, protein 238 is biotinylated and can be attached to a substrate through interaction with streptavidin.

Substrate 240 can include magnetic beads, fluorescent beads, silica coverslips, or microplates to attach amino acid-specific binder 200 to the substrate surface and can be a functionalized glass slide. Moreover, the substrate can be used for localization of amino acid-specific binder 200 by providing separation either by size or magnetism or physical movement of the substrate. The substrate can also be used to detect a taggant signal such as with fluorescent microscopy and can be a functionalized surface that is optically clear. Exemplary substrates 240 include NHS-ester functionalized glass slides, streptavidin coated magnetic beads or microplates, a nickel coated resin, and the like. In an embodiment, substrate 240 includes a nickel coated resin.

Chemical modifier 242 can include a reactive species that can be used in a non-covalent binding reaction or a cross-linking reaction or can be used to amplify a signal. Exemplary chemical modifiers 242 include click-chemistry compatible moieties, N-hydroxysuccinimide esters, biotin, maleimide, hydrazide, carbodiimide compounds for carboxylic acid cross-linking, photocatalysts, or electrocatalysts. In an embodiment, chemical modifier 242 includes an azide.

Exemplary taggant 214 are listed in Table 1 and can include a fluorescent moiety that can include embedded a fluorophore disposed in a shell, an electrochemical moiety, chemiluminescent moiety, Forster resonance energy transfer (FRET) pair, catalytic enzyme, chemical modification, or a combination comprising at least one of the foregoing moieties, that transduce or amplify stimulus 218 to a measurable response as taggant signal 216 for detecting a presence of amino acid-specific binder 200. In an embodiment, taggant 214 is a fluorophore (e.g. a fluorophore commercially available as ALEXAFLUOR such as ALEXAFLUOR647 and the like) that includes conjugated electrons to produce fluorescence upon stimulation by stimulant 218. Exemplary taggants 214 include horseradish peroxidase, fluorescein, rhodamine, and the like. In an embodiment, taggant 214 includes a fluorescently labelled dye (e.g., a dye such as commercially available as ATTOS32). Taggant 214 produces taggant signal 216 in response to being subjected to stimulus 218.

TABLE 1

| | Complex | | | |
|---|---|---|---|---|
| | formation | | | |
| Taggant | method | Stimulant | Signal | Detection |
| Fluorophore | NHS-ester lysine | Photon | Photon | intensity or |
| | sidechain | | | wavelength |
| Chemiluminescence | Luciferase fusion | ATP | Photon | Intensity |
| Electrochemiluminescence | Fusion with | Electrode | photon | PMT |
| | Ru(Bpy)3 | potential | | |
| FRET pair | a fluorophore on | Photon | Photon | Intensity or |
| | amino acid- | | | wavelength |
| | specific binder | | | |
| | 200, a | | | |
| | fluorophore on | | | |
| | analyte 212, or | | | |
| | fluorophores on | | | |
| | amino acid- | | | |
| | specific binder | | | |
| | 200 | | | |
| Catalytic enzyme | Horseradish | Addition of | Absorbance | Spectrophotometer |
| | peroxidase | chromogenic | at a | |
| | fusion | substrate | wavelength | |
| Radioactive element | ³⁵S-methionine, | None | Radioactivity | Scintillation |
| | ³²P- | | | counting or radio |
| | phosphorylation, | | | image |
| | or tritium | | | |
| | labeling of | | | |
| | amino acid | | | |
| | binder 200 | | | |

Stimulus 218 can include light emitted from a lamp, laser, LED, or a chromogenic substrate such as tetramethylbenzidine (TMB). Exemplary stimulus 218 includes laser light such as 30 mW, 488 nm laser light. In an embodiment, stimulus 218 is a photon, e.g., from a light source such as a laser, flash lamp, and the like. In an embodiment, stimulus 218 is a redox potential pulse.

Taggant signal 216 can have a temporal duration suitable for detection by an electrical amplifier, photodetector, scintillator, camera, and the like. In an embodiment, taggant signal 216 is fluorescence emission that is detected, e.g., by a detector such as a microscope that transmits the fluorescence to a CCD camera, wherein the location of emission can be correlated with the intensity of the signal.

In binder complex 228, with regard to indirect attachment of adjunct 236 to amino acid-specific binder 200, intervening member 246 can include a linker to connect adjunct 236 to amino acid-specific binder 200 but that does not provide additional functionality other than linking the two together. Intervening member 246 can be a protein, peptide, chemical moiety, nucleic acid, and the like. Moreover, intervening member 246 can be chemically inert such that it does not interfere with binding or signaling. Exemplary intervening members 264 include a poly-glycine or serine peptide, a polyethylene glycol (PEG), a glycan, an oligonucleotide, and the like. In an embodiment, intervening member 264 includes a GSGG peptide.

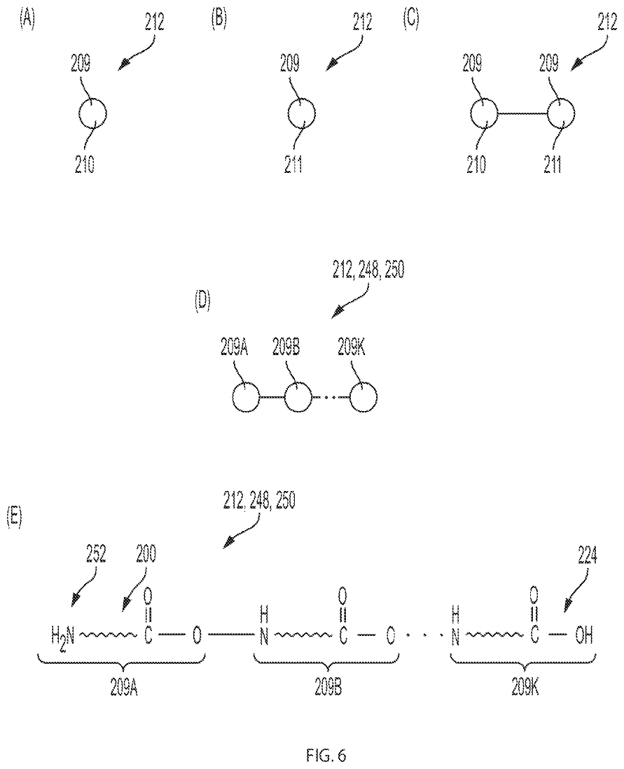

Amino acids 209 include binding amino acid 210 and analyte 212 as shown in FIG. 6. Here, in analyte 212, peptide 248 and protein 250 include a plurality of amino acids 209 (e.g., 209A, 209B . . . 209k) interconnected and terminating with N-terminal amino acid 220 that has free amine 252 and penultimate residue 234 and terminating with C-terminal end 224.

Amino acid-specific binder 200 selectively binds to binding amino acid 210 of analyte 212. Analyte 212 can include binding amino acid 210, non-binding amino acid 211, peptide 248, protein 250, or a combination thereof. Exemplary analytes 212 include proteins, peptides, free amino acids, and the like. In an embodiment, analyte 212 includes a protein that is cleaved using trypsin to produce a mixture of analytes 212 including binding amino acids 210 and non-binding amino acids 211.

Amino acid-specific binder 200 selectively binds to binding amino acid 210. Binding amino acid 210 can include certain naturally occurring amino acids, modified naturally occurring amino acids, non-naturally occurring amino acids, or modified non-naturally occurring amino acids. Selective binding of amino acid-specific binder 200 to binding amino acid 210 isolates binding amino acid 210 from other components in a fluid, identifies binding amino acid 210 as a particular species of amino acid (e.g., Leu, Phe, Trp, Tyr), and the like.

As used herein, “naturally occurring amino acid” refers to the 20 naturally occurring amino acids. Binding amino acids 210 that are naturally occurring amino acids are selected from group consisting of phenylalanine, tryptophan, tyrosine, leucine, isoleucine, and valine. As used herein, “modified naturally occurring amino acid” refers to naturally occurring amino acids in which a sidechain has been modified. Exemplary modifications include methylation, phosphorylation, glycosylation, deamination, oxidation, or selenocysteine formation. Accordingly, binding amino acids 210 that are modified naturally occurring amino acids include phosphotyrosine, N-acetylated valine, kynurenine and the like.

As used herein, “non-naturally occurring amino acid” refers to amino acids that are not naturally incorporated into peptide or protein polymers but can be synthetically incorporated into a polypeptide. Exemplary non-naturally occurring amino acids are D-amino acids, homo-amino acids, and amino acids with a non-natural sidechain such as biphenylalanine or azidophenylalanine. Accordingly, binding amino acids 210 that are non-naturally occurring amino acids include 5-bromo-tryptophan, homophenylalanine, homophenylalanine methyl ester hydrochloride, and the like.

As used herein, “modified non-naturally occurring amino acid” refers to a non-naturally occurring amino acid that has been modified. Exemplary modifications include such as methylation, phosphorylation, glycosylation, deamination, oxidation, or selenocysteine formation. Accordingly, binding amino acids 210 that are modified non-naturally occurring amino acids include 5-bromo-tryptophan, homophenylalanine, homopenylalanine methyl ester hydrochloride, and the like.

Amino acid-specific binder 200 does not bind to non-binding amino acid 211. Non-binding amino acid 211 can be a naturally occurring or non-naturally occurring amino acid exclusive of binding amino acid 210. Exemplary non-binding amino acids 211 include arginine, alanine, serine, threonine, proline, aspartic acid, asparagine, glutamine, glutamic acid. Since amino acid-specific binder 200 does not bind to non-binding amino acid 211 but does selectively bind to binding amino acid 210, non-binding amino acid 211 is determined as not belonging to the group of binding amino acids 210 selectively bound by amino acid-specific binder 200. Accordingly, while binding of amino acid-specific binder 200 to binding amino acid 210 can be used to isolate binding amino acid 210 from other components in a fluid, identify binding amino acid 210 as a particular species of amino acid (e.g., Leu, Phe, Trp, Tyr), and the like, not binding non-binding amino acid 211 can be used separate non-binding amino acid 211 from binding amino acid 210 and, by negative implication, determine a set of possible identities for binding amino acid 210.

Peptide 248 can include a plurality of amino acids, including binding amino acid 210, non-binding amino acid 211, or a combination thereof. Moreover, amino acids in peptide 248 are arranged to include N-terminal amino acid 220 and C-terminal end 224. Peptide 248 can be naturally occurring or can be a portion of a longer peptide or protein. Exemplary peptides 248 include a peptide from a proteolytic or tryptic digest of an isolated protein or protein found in blood or serum. Binding of amino acid-specific binder 200 to binding amino acid 210 can be used to isolate binding amino acid 210 from other components in a fluid, identify binding amino acid 210 as a particular species of amino acid (e.g., Leu, Phe, Trp. Tyr), and the like.

Protein 250 can include a plurality of amino acids, including binding amino acid 210, non-binding amino acid 211, or a combination thereof. Moreover, amino acids in protein 250 are arranged to include N-terminal amino acid 220 and C-terminal end 224. Protein 250 can be obtained from a mixture of proteins as found within a blood or serum sample. In an embodiment, protein 250 includes serum proteins.

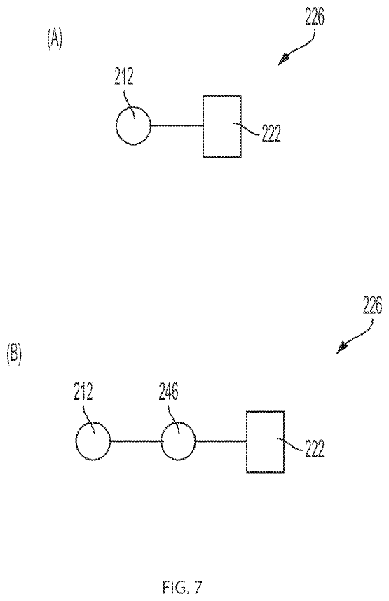

In some embodiments, with reference to FIG. 7, analyte 212 forms anchored analyte 226 in combination with anchor 222. Anchor 222 can include a substrate containing a surface on which to immobilize the analyte such that it can be sequestered or measured. Anchor 222 can be a resin, glass slide, magnetic bead. Exemplary anchor 222 includes a streptavidin coated sensor, microplate, and the like. In an embodiment, anchor 222 includes a streptavidin coated microplate, and intervening member 264 includes biotin.

Exemplary anchored analyte 226 includes a peptide analyte 212 anchored via the lysine sidechain to an NHS-ester coated glass slide and the like. It is contemplated that attachment of analyte 212 to anchor 222 can include a covalent bond, an ionic bond, electrostatic interaction (e.g., a π-cation interaction, dipole-dipole interaction, a multi-pole interaction, and the like), intercalation, a clathrate arrangement (e.g., with analyte 212 partially or wholly trapped in anchor 222 or vice-versa, such that N-terminal amino acid 220 or binding amino acid 210 is exposed to amino acid-specific binder 200 for selectively binding), and the like. Further, analyte 212 can be attached to anchor 222 either directly, indirectly, or a combination thereof. When analyte 212 is directly attached to anchor 222, direct attachment occurs in an absence of an intervening member between analyte 212 and anchor 222. When analyte 212 is indirectly attached to anchor 222, indirect attachment occurs in a presence of the intervening member 246 between analyte 212 and anchor 222.

In an embodiment, anchored analyte 226 includes a peptide analyte 212 anchored via the lysine sidechain to an NHS-ester coated glass slide and the like.

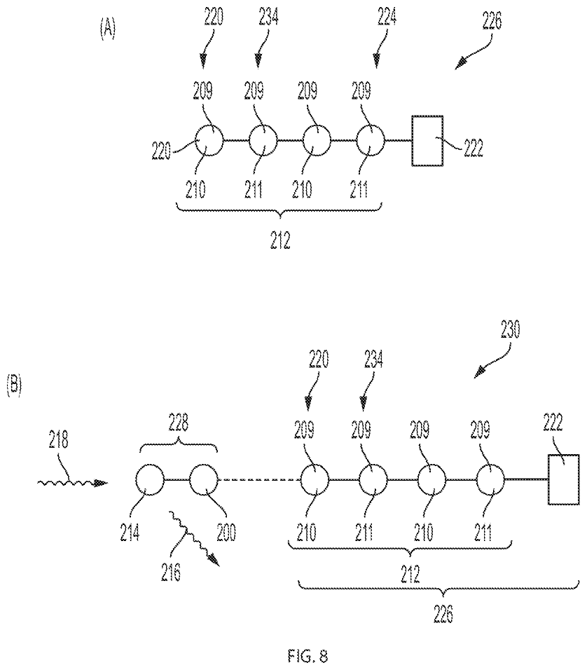

With reference to FIG. 8, selectively binding binder complex 228 to anchored analyte 226 forms tagged complex 230, e.g., to determine an identity of amino acid 209 in analyte 212 of anchored analyte 226. When analyte 212 is protein 250, amino acids in protein 250 can be sequenced using binder complex 228.

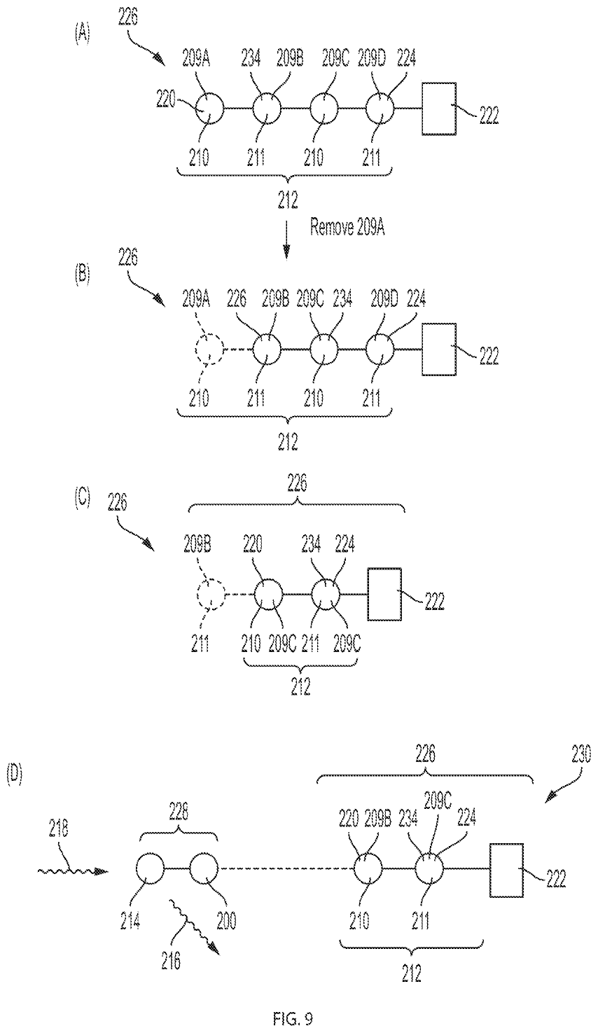

In determining a sequence of amino acids in analyte 212 in anchored analyte 226, with reference to FIG. 9, N-terminal amino acid 220 can be removed by chemical modification to expose the penultimate residue 234 as the new N-terminal amino acid 220 (panel D). Subsequent removal to expose the next penultimate residue 234 as the new N-terminal amino acid 220 can be repeated such that every new amino acid in analyte 212 can be sequentially subjected to binder complex 228 for sequencing.

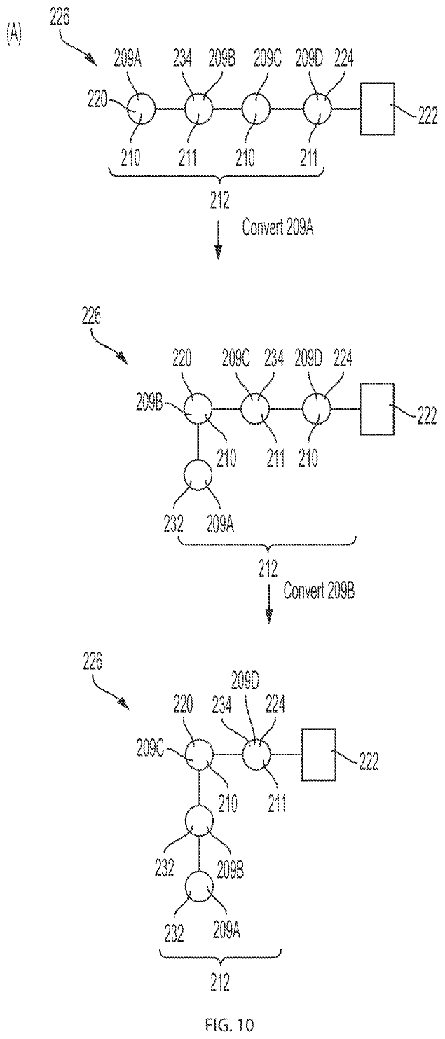

In determining a sequence of amino acids in analyte 212 in anchored analyte 226, with reference to FIG. 10, N-terminal amino acid 220 can be converted to inert residue 232. As used herein. “inert residue” refers to an amino acid that does not bind to amino acid-specific binder 200. The inert residue can be subsequently removed to expose the new penultimate residue 234 such that every new amino acid in analyte 212 can be sequentially subjected to binder complex 228 for sequencing.

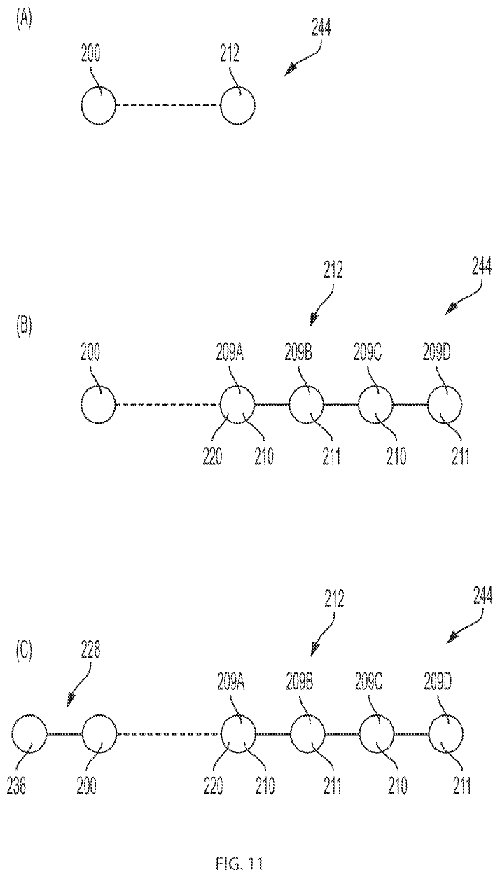

With reference to FIG. 11, selectively binding binder complex 228 to analyte 212, not in anchored analyte 226, forms isolation complex 244, e.g., to isolate analyte 212, to determine an identity of amino acid 209 in analyte 212 of isolation complex 244 and the like. Isolation complex 244 can be isolated from a heterogeneous composition containing analyte 212 using properties of isolation complex 244 such as the molecular weight. A difference in molecular weight between the isolation complex and undesired components in the composition must be great enough so that isolation complex 244 can be separated from other constituents in the composition by dialysis, chromatography, and the like.

Amino acid-specific binder 200 can be made in various ways. A process for making amino acid-specific binder 200 can include selecting a sequence for amino acid-specific binder 200 and expressing and purifying amino acid-specific binder 200 from an organism or by recombinant formation. A protein can be purified from the organism with a purification technique. Purification can include ion-exchange on a column that includes a cation-exchanger column or anion-exchanger column (e.g., diethylaminomethyl (DEAE) column), a mixed-mode ion exchanger (e.g., hydroxyapatite), or column that separates proteins based on hydrophobicity. A protein can be purified by size exclusion chromatography (e.g., gel-filtration) or in a density gradient (such as glycerol). Purification can be performed with binding to a different column that can include a specific chemical characterization of each protein. For recombinant expression in Escherichia coli, purification can be facilitated using a tag such as histidine, maltose binding protein (MBP), glutathione S-transferase (GST), and the like. A gene can be cloned into a pET15b vector with an additional His6-tag at an N-terminus of the protein, followed by a tobacco etch virus (TEV) protease cleavage site (MGHHHHHHENLYFQG (Sequence ID No. 13)), using the NcoI and XhoI restriction sites and expressed in BL21 E. coli cells. Expression from pET vector is induced with 0.5 mM IPTG when optical density at 600 nm (OD₆₀₀) reaches 1.0 absorbance units and further incubated for 6 hours at 37° C. or 16 hours at 15° C. Cells are harvested by centrifugation at 5000 g for 20 minutes, and cell pellets can be frozen. Frozen cell pellets are resuspended in a lysis buffer (e.g., 100 mM Tris-HCl, pH 8.0, 300 mM NaCl, 25 mM imidazole, or 50 mM sodium phosphate, 300 mM NaCl, or 20 mM Hepes, pH 8.0, 150 mM KCl) and sonicated on a 500 W sonicator with a C1334 probe at 20% amplitude for a time (e.g., 4 seconds on, 20 seconds off, for 90 minutes) that provides a selected total time (e.g., 15 minutes) of sonication. The lysate is centrifuged (e.g., at 20,000 g for 40 minutes) and then incubated (e.g., for one hour) that can include a chelating fast flow sepharose resin coated with nickel and pre-equilibrated in lysis buffer. The mixture is centrifuged (e.g., at 1000 g for 10 minutes) and supernatant removed, and the resin resuspended in lysis buffer that can be used to form a column. The column is washed with lysis buffer, wash buffer (e.g., lysis buffer with imidazole), and eluted with elution buffer. Protein that is eluted is subjected to dialysis into lysis buffer. Protein is removed from dialysis tubing and centrifuged, and the supernatant concentration measured by Bradford assay against a BSA standard curve. The protein is loaded onto a size exclusion chromatography column pre-equilibrated in lysis buffer. Fractions are collected from the size exclusion chromatography column and monitored at 280 nm, wherein absorption peaks are compared with a standard and analyzed by electrophoresis such as SDS-PAGE. Fractions are combined, concentrated by centrifugation with a molecular weight cutoff, such as 10 kDa, centrifuged, and measured by Bradford assay to prepare amino acid-specific binder 200.

In an embodiment, making binder complex 228 includes expressing a fusion protein of amino acid-specific binder 200 and adjunct protein 238 in an organism and purifying the fusion protein from the organism. In an embodiment, making binder complex 238 includes expressing a tagged variant of amino acid-specific binder such that it can be labeled with biotin during expression. The biotin contacts amino acid-specific binder 200 with substrate 240. In an embodiment, making binder complex 238 includes incubating the amino acid-specific binder 200 with an amine reactive chemical moiety such as NHS-ester HRP or taggant such as a fluorophore such as an NHS-ester fluorescein so that the amino-acid specific binder 200 lysine residues are linked to the fluorophore or chemical moiety.

Amino acid-specific binder 200 has numerous advantageous and unexpected benefits and uses. In an embodiment, with reference to FIG. 7 and FIG. 9, a process for selectively identifying N-terminal amino acid 220 includes providing analyte 212 including protein 250, peptide 248, amino acid 209, or a combination thereof, contacting C-terminal end 224 of analyte 212 with anchor 222; anchoring C-terminal end 224 to anchor 222 to form anchored analyte 226; contacting N-terminal amino acid 220 of anchored analyte 226 with binder complex 228, binder complex 228 include: amino acid-specific binder 200; and taggant 214 attached to amino acid-specific binder 200; selectively binding amino acid-specific binder 200 of binder complex 228 to N-terminal amino acid 220 of anchored analyte 226 when N-terminal amino acid 220 includes binding amino acid 210 to form tagged complex 230; subjecting taggant 214 of tagged complex 230 to stimulus 218; producing, by taggant 214 of tagged complex 230, taggant signal 216 in response to stimulus 218; detecting taggant signal 216; and identifying N-terminal amino acid 220 based on taggant signal 216, wherein amino acid-specific binder 200 binds selectively to binding amino acid 210.

In the process for selectively identifying N-terminal amino acid 220, providing analyte 212 includes purifying or extracting the analyte 212 from a mixture of components that may interfere with subsequent reactions. Exemplary purifications include high performance liquid chromatography (HPLC) or precipitation with ammonium sulfate. A protein can also be digested using a protease such as trypsin to create multiple peptides which can serve as analytes 212. An immobilized trypsin can be used to create multiple peptides by digestion of a protein or serum sample and purification of the peptides from the trypsin.

In the process for selectively identifying N-terminal amino acid 220, contacting C-terminal end 224 of analyte 212 with anchor 222 includes incubating or flowing the C-terminal end 224 of analyte 212 over the anchor 222.

In the process for selectively identifying N-terminal amino acid 220, anchoring C′-terminal end 224 to anchor 222 to form anchored analyte 226 includes incubating the C-terminal end 224 with anchor 222 under reaction conditions to covalently link the two. Exemplary reactions would include performing an N-hydroxysuccinimide (NHS)-ester reaction to link the C-terminal amino acid sidechain lysine within analyte 212 with anchor 222 that is modified with an NHS-ester to produce an amide bond.

In the process for selectively identifying N-terminal amino acid 220, contacting N-terminal amino acid 220 of anchored analyte 226 with binder complex 228 includes incubating anchored analyte 226 and binder complex 228 in a reaction buffer for a time (e.g., from 5 sec to 30 min) for the binding reaction to occur based on a binding affinity of amino acid-specific binder 200 under a set of binding conditions (e.g., in IX PBS at 30° C.). When N-terminal amino acid 220 is non-binding amino acid 211, the binding reaction does not occur.

In the process for selectively identifying N-terminal amino acid 220, selectively binding amino acid-specific binder 200 of binder complex 228 to N-terminal amino acid 220 of anchored analyte 226 when N-terminal amino acid 220 includes binding amino acid 210 includes incubating anchored analyte 226 and binder complex 228 in a reaction buffer for a time (e.g., from 5 sec to 30 min) for the binding reaction to occur based on a binding affinity of amino acid-specific binder 200 under a set of a binding conditions (e.g., in 1×PBS at 30° C.). When N-terminal amino acid 220 includes binding amino acid 210, the binding reaction occurs.

With reference to taggants and stimulants, signal, and detection listed in Table 1, in the process for selectively identifying N-terminal amino acid 220, subjecting taggant 214 of tagged complex 230 to stimulus 218 includes exposing tagged complex 230 on a fluorescent microscope that provides a select wavelength of light as a stimulant to produce taggant response, wherein an LED can produce excitation at 628 nm as a stimulus.

In the process for selectively identifying N-terminal amino acid 220, producing, by taggant 214 of tagged complex 230, taggant signal 216 in response to stimulus 218 includes, e.g., producing a fluorescent photon.

In the process for selectively identifying N-terminal amino acid 220, detecting taggant signal 216 includes detecting emission with a microscope that includes a detector that detects a selected wavelength of emission. e.g., 692 nm fluorescence.

In the process for selectively identifying N-terminal amino acid 220, identifying N-terminal amino acid 220 based on taggant signal 216 includes analyzing the signal response and interpreting the response based on the experimental design associated with the tagged binder complex 228. In an embodiment, the taggant is a fluorophore with a selected wavelength of emission response that provides a signal for detection through fluorescence intensity at a selected wavelength of the response to identity binding amino acid 210.

With reference to FIG. 9 and FIG. 10, the process for selectively identifying N-terminal amino acid 220, also can include removing N-terminal amino acid 220 from anchored analyte 226 so that penultimate residue 234 becomes N-terminal amino acid 220 of anchored analyte 226 by Edman degradation.

The process for selectively identifying N-terminal amino acid 220, also can include contacting N-terminal amino acid 220 of anchored analyte 226 with binder complex 228 by incubating anchored analyte 226 and binder complex 228 in a reaction buffer for a time (e.g., from 5 sec to 30 min) for the binding reaction to occur based on a binding affinity of amino acid-specific binder 200 under a set of a binding conditions (e.g., in 1×PBS at 30° C.). When N-terminal amino acid 220 includes non-binding amino acid 211, the binding reaction does not occur.

The process for selectively identifying N-terminal amino acid 220, also can include selectively binding amino acid-specific binder 200 of binder complex 228 to N-terminal amino acid 220 of anchored analyte 226 when N-terminal amino acid 220 is binding amino acid 210 to form tagged complex 230 by incubating anchored analyte 226 and binder complex 228 in a reaction buffer for a time (e.g., from 5 sec to 30 min) for the binding reaction to occur based on a binding affinity of amino acid-specific binder 200 under a set of binding conditions (e.g., in 1×PBS at 30° C.). When N-terminal amino acid 220 includes binding amino acid 210, the binding reaction occurs, and the tagged complex forms.

The process for selectively identifying N-terminal amino acid 220, also can include subjecting taggant 214 of tagged complex 230 to stimulus 218. In an embodiment, tagged complex 230 is exposed to a selected wavelength and intensity of light to excite the fluorophore. In an embodiment, subjecting taggant 214 of tagged complex 230 to stimulus 218 includes adding a chromogenic substrate. Table 1 lists a taggant, stimulant, signal, and detection for adjuncts shown in Table 2.

The process for selectively identifying N-terminal amino acid 220 also can include producing, by taggant 214 of tagged complex 230, taggant signal 216 in response to stimulus 218. In an embodiment, taggant 214 is a fluorophore that emit light as taggant response at an emission wavelength after being stimulated by an excitation wavelength as the stimulus. In an embodiment, chromogenic substrate produces a chromogenic signal as when contacted by HRP as taggant 214.

The process for selectively identifying N-terminal amino acid 220 also can include detecting taggant signal 216 by methods listed in Table 2 for each taggant. In an embodiment, detection can involve a microscope with a CCD camera and selected filters in an optical system that detects a wavelength of emitted light. In an embodiment, a spectrophotometer measures absorbance at a selected wavelength to detect a chromogenic substrate. In an embodiment, a scintillation counter measures radioactivity of a radiolabeled complex.

TABLE 2

| Adjunct | Isolation Technique | Separation Property |
|---|---|---|
| High molecular weight | Dialysis | Size |
| protein | | |
| High molecular weight | Ultracentrifugation | Size |
| protein | | |
| Substrate | Physical Separation | binding analytes |
| | | are anchored |
| Protein with different | Precipitation | Solubility or |
| solubility | | Molecular weight |
| Protein with different | Isoelectric Gradient | Isoelectric point |
| isoelectric point | | |
| Protein with different | Density Gradient | Density |
| densities | | |

The process for selectively identifying N-terminal amino acid 220, also can include identifying N-terminal amino acid 220 based on taggant signal 216 by analyzing the signal response and interpreting the response based on tagged binder complex 228. When taggant 214 is a fluorophore, the intensity and wavelength of the taggant response identifies a binding amino acid 210 due to a higher signal than non-binding amino acid 211.

With reference to FIG. 10, instead of or in combination with removing N-terminal amino acid 220, the process for selectively identifying N-terminal amino acid 220 also can include converting N-terminal amino acid 220 to inert residue 232 by performing a partial Edman degradation reaction so that phenylisothiocyanate (PITC) remains attached to the N-terminal amino acid such that a binding reaction does not occur.

The process for selectively identifying N-terminal amino acid 220, also can include converting penultimate residue 234 to N-terminal amino acid 220 when inert residue 232 is removed by continuing the Edman degradation reaction to remove the PITC.

The process for selectively identifying N-terminal amino acid 220, also can include contacting N-terminal amino acid 220 of anchored analyte 226 with binder complex 228 by incubating anchored analyte 226 and binder complex 228 in a reaction buffer for a time (e.g., from 5 sec to 30 min) for the binding reaction to occur based on a binding affinity of amino acid-specific binder 200 under a set of a binding conditions (e.g., in 1×PBS at 30° C.′). When N-terminal amino acid 220 includes binding amino acid 210, the binding reaction occurs, and the tagged complex forms.

The process for selectively identifying N-terminal amino acid 220 also can include selectively binding amino acid-specific binder 200 of binder complex 228 to N-terminal amino acid 220 of anchored analyte 226 when N-terminal amino acid 220 is binding amino acid 210 to form tagged complex 230 by incubating anchored analyte 226 and binder complex 228 in a reaction buffer for a time (e.g., from 5 sec to 30 min) for the binding reaction to occur based on a binding affinity of amino acid-specific binder 200 under a set of binding conditions (e.g., in 1×PBS at 30° C.). When N-terminal amino acid 220 includes binding amino acid 210, the binding reaction occurs and tagged complex 230 forms.

The process for selectively identifying N-terminal amino acid 220, also can include subjecting taggant 214 of tagged complex 230 to stimulus 218 by exposing tagged complex 230 to a selected wavelength and intensity of light to excite the fluorophore. In an embodiment, subjecting taggant 214 of tagged complex 230 to stimulus 218 includes adding chromogenic substrate.

The process for selectively identifying N-terminal amino acid 220 also can include producing, by taggant 214 of tagged complex 230, taggant signal 216 in response to stimulus 218, e.g., by a method listed in Table 1. In an embodiment, detection can include detecting taggant response with a microscope including a CCD camera and filters in an optical system to detect a wavelength of emitted light. In an embodiment, a spectrophotometer measures absorbance at a selected wavelength to detect a chromogenic substrate. In an embodiment, a scintillation counter measures radioactivity of a radiolabeled complex.

The process for selectively identifying N-terminal amino acid 220, also can include detecting taggant signal 216. The process for selectively identifying N-terminal amino acid 220, also can include identifying N-terminal amino acid 220 based on taggant signal 216. In the process, converting N-terminal amino acid 220 to inert residue 232 can include chemically changing N-terminal amino acid 220 prior to producing inert residue 232.

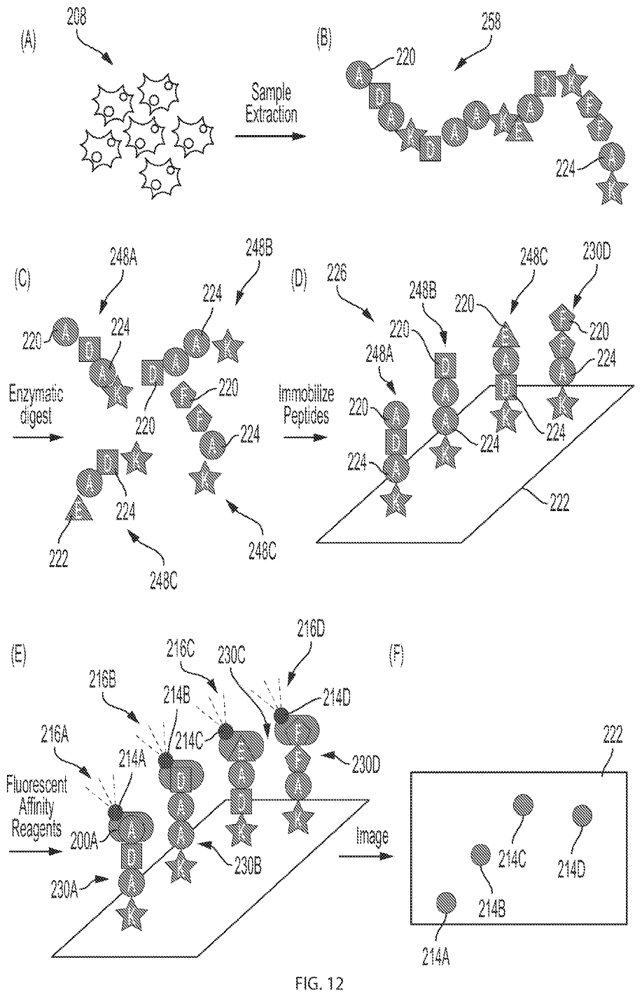

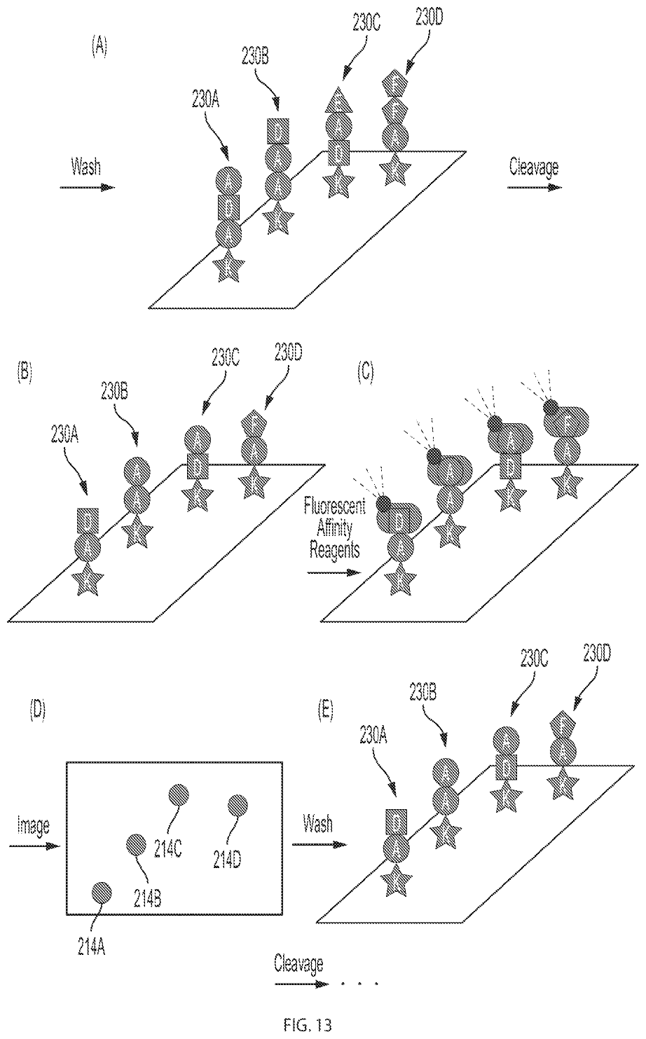

In an embodiment, with reference to FIG. 12 and FIG. 13, a process for sequencing protein 250 includes providing sample 208; extracting protein 250 from sample 208; enzymatically digesting protein 250 to provide a plurality of peptides 248 (e.g., 248A, 248D); forming anchored analyte 226 by immobilizing peptides 248 on anchor 222; producing tagged complex 230; detecting taggant signal 216 from taggant 214; removing binder complex 228 by washing; cleaving N-terminal amino acid 220 so that penultimate residue 234 becomes a new N-terminal amino acid 220; and repeating so that multiple repetitions of steps occur, including producing tagged complex 230; detecting taggant signal 216 from taggant 214; and removing binder complex 228 by washing; cleaving N-terminal amino acid 220 so that penultimate residue 234 becomes a new N-terminal amino acid 220 to sequence protein 250.

In the process, chemically changing N-terminal amino acid 220 prior to producing inert residue 232 can include phosphorylating free amine 252 of N-terminal amino acid 220.

In an embodiment, with reference to FIG. 11, a process for selectively isolating analyte 212 includes contacting amino acid-specific binder 200 with analyte 212 by incubating amino acid-specific binder with the analyte or analyte within a mixture in a reaction buffer for a time for binding to occur if the analyte includes binding amino acid 210. The process includes selectively binding amino acid-specific binder 200 to N-terminal amino acid 220 of analyte 212 when N-terminal amino acid 220 includes binding amino acid 210 to form isolation complex 244 by incubating them in a reaction buffer for a time for binding to occur. The process also includes separating, e.g., by dialysis, isolation complex 244 from a fluid in which isolation complex 244 is disposed to selectively isolate analyte 212.

In the process for selectively isolating analyte 212, separating isolation complex 244 from the fluid can include separating isolation complex 244 based on a size of isolation complex 244 relative to a size of other constituents in fluid by dialysis in which the isolation complex is too large to move through a pore but other constituents that the complex is being isolated from communicate through the pore. In an embodiment, the isolation complex is an immunoglobulin fusion, and analyte 212 is phenylalanine. The complex can be isolated by dialysis through, e.g., a 10 kDa molecular weight cutoff membrane. Separating can include precipitating isolation complex 244 from the fluid, ultracentrifuging in a glycerol gradient and separating the gradient fractions, and the like.

Amino acid-specific binder 200 and processes disclosed herein have numerous beneficial uses, including protein sequencing, peptide fingerprinting, and isolating amino acid analytes. Advantageously, amino acid-specific binder 200 overcomes limitations or technical deficiencies of conventional articles such as the selectivity or specificity for a particular amino acid over similar amino acids. Additionally, amino acid-specific binder 200 has higher affinity combined with high specificity than conventional approaches. As such, amino acid-specific binder 200 can discriminate amino acids for sequencing. Amino acid-specific binder 200 is specific for an N-terminal amino acid rather than an internal residue containing the same amino acid sidechain. Accordingly, amino acid-specific binder 200 determines the identity of the amino acid and a position of the amino acid in a peptide or protein. Additionally, amino acid-specific binder 200 binds a binding amino acid that is not part of a protein or peptide. To circumvent an inability to determine a position of an amino acid (N-terminal, internal, or C-terminal), conventional sequencing or fingerprinting methods rely on a loss of signal if the amino acid being detected is removed from the peptide and interpret a loss of signal at a particular iteration as an indication of position or register within the peptide. A detection scheme that relies on loss of signal is limited when another factor (such as quenching of a signaling fluorophore) also results in a loss of signal. Amino acid-specific binder 200 and tagged complex described herein overcome this limitation by adding a new amino acid-specific binder in each iteration of the sequencing process. By tagging amino acid-specific binder 200 rather than tagging the analyte directly, embodiments herein provide greater signal control and a more universal approach than conventional technology. Embodiments herein produce or detect different types of signals and include a plurality of detection schemes that provide enhanced flexibility in types of substrates and different contexts in which analyte are manipulated.

Amino acid-specific binder 200 and processes herein unexpectedly isolate analytes from a composition that includes the analyte disposed in a fluid that also includes other constituents such as amino acids, proteins, and the like. Due to the ability to anchor either the analyte or the amino acid-specific binder, the reagent can be used in many different separation processes in addition to sequencing processes.

The articles and processes herein are illustrated further by the following Example, which is non-limiting.

### EXAMPLE

### Enhanced N-terminal Amino Acid Binding

Using available scientific literature, it was found that Synechococcus elongatus ClpS demonstrates a preference for binding to peptides with an N-terminal leucine. The distinct sequence features of S. elongatus ClpS were discerned by comparison to other well-characterized ClpS variants and, using the publicly available Pfam database, a list of ClpS variants with sequence features like those of S. elongatus ClpS was compiled Thermosynechococcus elongatus ClpS2 was selected from this list as the variant of greatest experimental interest, due to ease of handling proteins from thermophilic organisms.

With regard to plasmid construction, wild-type T. elongatus ClpS2 gene was synthesized by GeneArt and cloned into the pET15b vector with an additional His6-tag at the N-terminus of the protein, followed by a Tobacco Etch virus (TEV) protease cleavage site, using the NcoI and XhoI restriction sites.

With regard to protein purification, the wild-type T. elongatus ClpS2 proteins were expressed in BL21 Escherichia coli cells. Expression was induced with 0.5 mM IPTG when the OD₆₀₀ reached 1.0 and incubated for 16-18 hours at 20° C. The cells were harvested by centrifugation at 5000 g for 20 minutes, and the cell pellets frozen for future use.

Frozen cell pellets were resuspended in phosphate-buffered saline (pH 7.2) and sonicated with a probe at 20% amplitude for 4 seconds on, 20 seconds off, for 90 minutes, which results in 15 minutes total sonication time. Sonicated material was heated to 60° C. for sixty minutes, then cooled on ice for fifteen minutes. The lysate was centrifuged at 20000 g for 30 minutes and then incubated for one hour with chelating fast flow sepharose resin coated with nickel and pre-equilibrated in lysis buffer. The resin was separated from the lysate by collection on a gravity-flow column. The column was then washed with 10 column volumes (CVs) of PBS, and then S CVs of wash buffer (PBS+50 mM imidazole), before eluting with 5 CVs of elution buffer (PBS+250 mM imidazole). The eluted protein was then loaded onto a S200 26/60 size exclusion chromatography column pre-equilibrated in 2CV of 20 mM HEPES. pH 8. 5 mL Fractions were collected and tracked at 280 nm; peaks were compared with a gel filtration standard and further analyzed by SDS-PAGE. Fractions were combined, concentrated by ultra-centrifugation with a 10 kDa MWCO, centrifuged for 40 minutes at 20,000 g, and measured by the Nanodrop assay.

The initial binding activity of T. elongatus ClpS2 was characterized using bio-laver interferometry (BLI) In BLI, a functionalized fiber-optic probe is placed in a solution of analyte and the accumulation of analyte on the functional group of the probe is measured by changes in reflected light within the probe. A BLI probe functionalized with streptavidin is allowed to accumulate biotinylated peptide. Probes with peptide are then placed in solutions of ClpS of varying concentration and allowed to equilibrate. BLI response is then graphed to concentration to establish a binding curve. Dilutions of T. elongatus ClpS2 were made in 20 mM HEPES pH 8, 150 mM KCl, 0.1% BSA, and 0.05% Tween-20 at concentrations of 0.312, 0.625, 1.25, 2.5, 5, 10, 20, and 40 μM. Comparison across four peptides that vary only by their N-terminal amino acid show a strong preference for binding leucine.

The gene was cloned into the pCTCON2 vector for yeast display by amplifying the gene by polymerase chain reaction (PCR) with primers to add the NheI and BamHI sites, and then ligating this to the vector such that the gene is located to the C-terminal end of the aga2-encoding gene, separated by a factor XA cleavage site, an HA-tag and a [GGGGS (Sequence ID No. 14)]×3 spacer. There is also a C-terminal myc-tag before the stop codon. A vector was also created that contains no gene insert, but rather a SacII site and a SpeI site, so that the vector can be linearized at this site for homologous recombination.

With regard to random mutagenesis library creation, to create the random mutagenesis libraries, error-prone PCR was used. The gene encoding the ClpS protein of interest was amplified using the HA-tag for (CCATACGACGTTCCAGACTAC (Sequence ID No. 15) and T7 (TAATACGACTCACTATAGGG (Sequence ID No. 16) primers in a reaction containing 0.2 mM dATP, 1 mM dCTP, 0.2 mM dGTP, 1 mM dTTP, 10 mM MgCl₂, and 0.5 mM MnCl₂, 1×Taq reaction buffer (20 mM Tris-HCl, pH 8.4, 50 mM KCl) without MgCl₂, and Taq DNA polymerase. The PCR product was used for homologous recombination, as described below. The libraries each contained at least 1 million naïve members, and greater than 50% of the colonies contained at least one mutation from the ten colonies sequenced.

With regard to Saccharomyces cerevisiae (yeast) transformation. EBY100 strain S. cerevisiae were transformed with pCTCON2 plasmids containing the wild-type ClpS genes using the Frozen EX Yeast Transformation II kit and subsequently grown on selective media as the pCTCON2 plasmid harbors the ability to synthesize tryptophan. Synthetic dextrose media supplemented with casamino acids lacking tryptophan (SD-CAA) and containing 100 μg/mL ampicillin was used to grow all the yeast used in this study. Protein surface expression was induced by resuspending the cells in synthetic galactose media supplemented with casamino acids lacking tryptophan (SG-CAA).

With regard to site-saturation library creation/homologous recombination in yeast, EBY100 S. cerevisiae cells were grown overnight to an OD₆₀₀ of 3 in YPD media at 30° C. This was used to inoculate a 100 mL culture of YPD to OD₆₀₀ 0.3. After 5 hours, when cells had grown to OD₆₀₀ 1.0, the cells were transferred to 50 ml conical tubes and centrifuged at 3000 g for 3 min at 4° C. The cell pellet was washed twice with 50 mL ice cold sterile water and then washed once with 50 mL ice cold electroporation buffer (1M sorbitol/1 mM CaCl₂)). The cells were conditioned for electroporation by resuspending the cell pellet in 20 mL 0.1 M LiAc, 10 mM DTT and shaking for 30 min at 30 degrees. The cells were centrifuged as above and washed with 25 mL per tube of electroporation buffer before being resuspended in 200 μL of electroporation buffer to reach a final volume of about 1 mL. Cells were kept on ice until electroporation.

For electroporation, 400 μL of competent cells prepared as above were incubated with the vector and insert, in a 1:3 ratio, and kept on ice for 5 minutes. The vector used was the pCTCON2 plasmid described above containing the clpS gene of interest and digested within the clpS gene with the restriction enzymes NheI and BamHI. The insert used was the error prone PCR library obtained as described above. The cells were then transferred to a 0.2 cm electroporation cuvette and electroporated on the pre-set yeast settings (1.5 kV, 25 μF). The cells were transferred to a tube containing 4 mL of YPD media and 4 mL of 1 M sorbitol and incubated at 30° C. for 1 hour, 225 rpm. The cells were then centrifuged and resuspended in SD-CAA media and dilutions were plated to calculate library size, and the rest was grown in a flask containing 250 mL of SD-CAA media and passaged once before selections or sorting.

With regard to library selection, fluorescence-activated cell sorting (FACS) was performed using slightly modified protocols from the 2003 Pacific Northwest National Lab Yeast Display ScFV Antibody Library User's Manual and 2004 Methods in Molecular Biology Flow Cytometry Protocols. Yeast displaying a library of mutant ClpS proteins were grown in SD-CAA media overnight at 30° C. until the OD₆₀₀ was approximately 4.0. The yeast was used to inoculate a fresh culture at an OD₆₀₀ of 1.0 in a mixture of 80% SG-CAA/20% SD-CAA and incubated for 24 hours at 20° C. Approximately 109 yeast were washed and resuspended in 1 mL of Dulbecco's phosphate buffered saline containing 0.5% bovine serum albumin (PBS/BSA) (DPBS) containing 100 nM biotinylated peptide at room temperature for 1 hour. The yeast was pelleted by centrifugation at 3000 g for 2 minutes. The cells were then incubated with streptavidin-R-phycoerythrin (PE), and anti-myc AF647 for 30 minutes on ice. A typical reaction contained 1 mL of cells (containing approximately 10⁷ cells), peptide at a concentration of 100 nM, and 25 μL of a master mix containing 2 μL of an anti-myc antibody, 4 μL of the SAPE (streptavidin, R-phycoerythrin conjugate-1 mg/mL) and 19 μL of PBS/BSA for each sample.

Cells were washed with PBS/BSA, sorted using a FACS Sony SH800 cytometer, and collected in 1 mL of SD-CAA media. The threshold for collection was based on background levels of anti-myc AF647 and streptavidin-R-PE by cells in the absence of peptide. Cells which showed greater than background binding for both metrics in the presence of peptide are grouped in Q2. Q2 Cells were grown out and used to repeat the selection procedure. The number of cells that bound the peptide improved with each round. After three rounds of selection, not only is the count of Q2 cells higher for peptides with leucine, but it did not markedly change for other peptides shown to also bind to T. elongatus ClpS2.

Cells from the library after three rounds of sorting were lysed by shaking with glass beads. The DNA from the lysate was purified by phenol-chloroform extraction and ethanol precipitation, then the available ClpS genes were amplified using the primers from the previous error-prone PCR. The linear PCR product was sent for Illumina deep sequencing. This produced ˜83,000 sequence reads with 8467 unique amino acid sequences. Based on these sequences, mutational hotspots in the sequence of T. elongatus ClpS2 were identified. For each hotspot, the most statistically prevalent amino acid was identified. The combination of these most prevalent hotspot mutants is predicted to produce a ClpS variant with improved leucine binding activity and selectivity.

With regard to peptides, all peptides are named by indicating the first two residues, with the full sequence available in Table 3. All of the peptides had the same sequence for the C-terminal end of the peptide, XXAVECK (Sequence ID No. 18), where the N-terminal amino acids are varied. The peptides also contained a biotin linked via the lysine side chain on the C-terminal residue. X1G2 peptides started from a lyophilized form. Peptides were resuspended in 1×DPBS and diluted to the appropriate concentration into the experiment buffer. The peptides were synthesized in-house on a 20 μmol scale on a peptide synthesizer with amino acid reagents and biotin resin. Synthesis resulted in a peptide with an ethylene diamine spacer and then the biotin moiety. The peptide was then cleaved from the resin using 3 mL of trifluoroacetic acid (TFA), phenol, water, and triisopropylsilane (TIPS) in an 88:5:5:2 ratio. The peptide was subsequently rinsed with ice cold ether, pelleted by centrifugation at 4500 g for 10 min at 4° C. and decanted three times. It was then dried under nitrogen overnight at room temperature and subsequently lyophilized and stored at −20° C. until resuspension in the assay buffer.

### EMBODIMENTS INCLUDE

1. A binder complex for selectively identifying an amino acid, the binder complex comprising:
an amino acid-specific binder; and
an adjunct attached to the amino acid-specific binder,
wherein the amino acid-specific binder binds selectively to a binding amino acid, and
the amino acid-specific binder comprises an amino acid sequence comprising:
   a first amino acid sequence comprising X1-GQQVT-X2-QHVV-X3-K-X4-MI-X5-GGR-X6-E-X7-Y-X8-E;
   a second amino acid sequence comprising X1-RQQDT-X2-RHVA-X3-M-X4-MT-X5-EGQ-X6-E-X7-Y-X8-E;
   a third amino acid sequence comprising X1-RQQVA-X2-RHVA-X3-M-X4-MT-X5-EGQ-X6-V-X7-Y-X8-G;
   a fourth amino acid sequence comprising X1-RQQDT-X2-RHVA-X3-M-X4-KT-X5-EGQ-X6-E-X7-Y-X8-E;
   a fifth amino acid sequence comprising X1-RQQVT-X2-RHVA-X3-M-X4-MT-X5-KGQ-X6-E-X7-N-X8-E;
   a sixth amino acid sequence comprising X1-RQQVA-X2-RHVA-X3-M-X4-MT-X5-EGQ-X6-E-X7-Y-X8-E;
   a seventh amino acid sequence comprising X1-RQQDT-X2-RHVA-X3-M-X4-MT-X5-KGQ-X6-V-X7-Y-X8-G; or
   an eighth amino acid sequence comprising X1-RQQDT-X2-RHVA-X3-M-X4-MT-X5-EGQ-X6-V-X7-Y-X8-G;
   wherein;
   X1 comprises an amino acid sequence comprising ASVVPQE (Sequence ID No. 20);
   X2 comprises an amino acid sequence comprising

(Sequence ID No. 21
RKHYPNYKVIVLNDDFNTF;

X3 comprises an amino acid sequence comprising ACL (Sequence ID No. 22);
X4 comprises an amino acid sequence comprising KYIPN (Sequence ID No. 23);
X5 comprises an amino acid sequence comprising SDRAWELTNQVHY (Sequence ID No. 24);
X6 comprises an amino acid sequence comprising AIVWVGPQ (Sequence ID No. 25);
X7 comprises an amino acid sequence comprising QAEL (Sequence ID No. 26); and
X8 comprises an amino acid sequence comprising HEQLLRAGLTMAPLEP (Sequence ID No. 27),
such that a total percentage amount of substitutions and deletions to X1, X2, X3, X4, X5, X6, X7, and X8 is from 0% to less than 30%, exclusive of

(Sequence ID No. 28)
PSVVPQERQQVTRKHYPNYKVIVLNDDFNTFQHVAACLMKYIPNMTSDR
AWELTNQVHYEGQAIVWVGPQEQAELYHEQLLRAGLTMAPLEPE.

2. The binder complex of embodiment 1, wherein the amino acid sequence comprises:

(Sequence ID No. 1)
ASVVPQEGQQVTRKHYPNYKVIVLNDDFNTFQHVVACLKKYIPNMISDR
AWELTNQVHYGGRAIVWVGPQEQAELYHEQLLRAGLTMAPLEPE;
(Sequence ID No. 2)
ASVVPQERQQDTRKHYPNYKVIVLNDDENTFRHVAACLMKYIPNMTSDR
AWELTNQVHYEGQAIVWVGPQEQAELYHEQLLRAGLTMAPLEPE;
(Sequence ID No. 3)
ASVVPQERQQVARKHYPNYKVIVLNDDFNTFRHVAACLMKYIPNMTSDR
AWELTNQVHYEGQAIVWVGPQVQAELYHEQLLRAGLTMAPLEPG;
(Sequence ID No. 4)
ASVVPQERQQDTRKHYPNYKVIVLNDDFNTFRHVAACLMKYIPNKTSDR
AWELTNQVHYEGQAIVWVGPQEQAELYHEQLLRAGLTMAPLEPE;
(Sequence ID No. 5)
ASVVPQERQQVTRKHYPNYKVIVLNDDENTFRHVAACLMKYIPNMTSDR
AWELTNQVHYKGQAIVWVGPQEQAELNHEQLLRAGLTMAPLEPE;
(Sequence ID No. 6)
ASVVPQERQQVARKHYPNYKVIVLNDDFNTFRHVAACLMKYIPNMTSDR
AWELTNQVHYEGQAIVWVGPQEQAELYHEQLLRAGLTMAPLEPE;
(Sequence ID No. 7)
ASVVPQERQQDTRKHYPNYKVIVLNDDFNTFRHVAACLMKYIPNMTSDR
AWELTNQVHYKGQAIVWVGPQVQAELYHEQLLRAGLTMAPLEPG;
(Sequence ID No. 8)
ASVVPQERQQDTRKHYPNYKVIVLNDDFNTFRHVAACLMKYIPNMTSDR
AWELTNQVHYEGQAIVWVGPQVQAELYHEQLLRAGLTMAPLEPG;
(Sequence ID No. 9)
ASVVPQEGQQVTRKHYPNYKVIVLNDDFNTFQHVVACLKKYIPNMISDR
AWELTNQVHYGGRAIVWVGPQEQAELNHEQLLRAGLTMAPLEPE;
or
(Sequence ID No. 10)
ASVVPQERQQVARKHYPNYKVIVLNDDFNTFRHVAACLMKYIPNMTSDR
AWELTNQVHYEGQAIVWVGPQVQAELNHEQLLRAGLTMAPLEPE.

3. The binder complex of embodiment 1, wherein the adjunct comprises a taggant, a protein, a substrate, or a chemical modifier.
4. The binder complex of embodiment 3, wherein the taggant comprises:
a fluorescent moiety, an electrochemical moiety, or a combination comprising at least one of the foregoing moieties, and the taggant produces a taggant signal in response to receiving a stimulus.
5. A process for selectively identifying an N-terminal amino acid, the process comprising:
providing an analyte comprising a protein, a peptide, an amino acid, or a combination comprising at least one of foregoing;
contacting a C-terminal end of the analyte with an anchor;
anchoring the C-terminal end to the anchor to form an anchored analyte;
contacting an N-terminal amino acid of the anchored analyte with a binder complex, the binder complex comprising:
   an amino acid-specific binder; and
   a taggant attached to the amino acid-specific binder;
selectively binding the amino acid-specific binder of the binder complex to the N-terminal amino acid of the anchored analyte when the N-terminal amino acid comprises a binding amino acid to form a tagged complex;
subjecting the taggant of the tagged complex to a stimulus;
producing, by the taggant of the tagged complex, a taggant signal in response to the stimulus;
detecting the taggant signal; and
identifying the N-terminal amino acid based on the taggant signal,
wherein the amino acid-specific binder binds selectively to the binding amino acid, and
the amino acid-specific binder comprises an amino acid sequence comprising:
a first amino acid sequence comprising X1-GQQVT-X2-QHVV-X3-K-X4-MI-X5-GGR-X6-E-X7-Y-X8-E;
a second amino acid sequence comprising X1-RQQDT-X2-RHVA-X3-M-X4-MT-X5-EGQ-X6-E-X7-Y-X8-E;
a third amino acid sequence comprising X1-RQQVA-X2-RHVA-X3-M-X4-MT-X5-EGQ-X6-V-X7-Y-X8-G;
a fourth amino acid sequence comprising X1-RQQDT-X2-RHVA-X3-M-X4-KT-X5-EGQ-X6-E-X7-Y-X8-E;
a fifth amino acid sequence comprising X1-RQQVT-X2-RHVA-X3-M-X4-MT-X5-KGQ-X6-E-X7-N-X8-E;
a sixth amino acid sequence comprising X1-RQQVA-X2-RHVA-X3-M-X4-MT-X5-EGQ-X6-E-X7-Y-X8-E;
a seventh amino acid sequence comprising X1-RQQDT-X2-RHVA-X3-M-X4-MT-X5-KGQ-X6-V-X7-Y-X8-G; or
an eighth amino acid sequence comprising X1-RQQDT-X2-RHVA-X3-M-X4-MT-X5-EGQ-X6-V-X7-Y-X8-G;
wherein:
X1 comprises an amino acid sequence comprising ASVVPQE (Sequence ID No. 20);
X2 comprises an amino acid sequence comprising RKHYPNYKVIVLNDDFNTF (Sequence ID No. 21;
X3 comprises an amino acid sequence comprising ACL (Sequence ID No. 22);
X4 comprises an amino acid sequence comprising KYIPN (Sequence ID No. 23);
X5 comprises an amino acid sequence comprising SDRAWELTNQVHY (Sequence ID No. 24);
X6 comprises an amino acid sequence comprising AIVWVGPQ (Sequence ID No. 25);
X7 comprises an amino acid sequence comprising QAEL (Sequence ID No. 26); and
X8 comprises an amino acid sequence comprising HEQLLRAGLTMAPLEP (Sequence ID No. 27),
   such that a total percentage amount of substitutions and deletions to X1, X2, X3, X4, X5, X6, X7, and X8 is from 0% to less than 30%, exclusive of

(Sequence ID No. 28)
PSVVPQERQQVTRKHYPNYKVIVLNDDFNTFQHVAACLMKYIPNMTSDR
AWELTNQVHYEGQAIVWVGPQEQAELYHEQLLRAGLTMAPLEPE.

6. The process of embodiment 5, wherein the amino acid sequence comprises:

(Sequence ID No. 1)
ASVVPQEGQQVTRKHYPNYKVIVLNDDFNTFQHVVACLKKYIPNMISDR
AWELTNQVHYGGRAIVWVGPQEQAELYHEQLLRAGLTMAPLEPE;
(Sequence ID No. 2)
ASVVPQERQQDTRKHYPNYKVIVLNDDENTFRHVAACLMKYIPNMTSDR
AWELTNQVHYEGQAIVWVGPQEQAELYHEQLLRAGLTMAPLEPE;
(Sequence ID No. 3)
ASVVPQERQQVARKHYPNYKVIVLNDDFNTFRHVAACLMKYIPNMTSDR
AWELTNQVHYEGQAIVWVGPQVQAELYHEQLLRAGLTMAPLEPG;
(Sequence ID No. 4)
ASVVPQERQQDTRKHYPNYKVIVLNDDFNTFRHVAACLMKYIPNKTSDR
AWELTNQVHYEGQAIVWVGPQEQAELYHEQLLRAGLTMAPLEPE;
(Sequence ID No. 5)
ASVVPQERQQVTRKHYPNYKVIVLNDDENTFRHVAACLMKYIPNMTSDR
AWELTNQVHYKGQAIVWVGPQEQAELNHEQLLRAGLTMAPLEPE;
(Sequence ID No. 6)
ASVVPQERQQVARKHYPNYKVIVLNDDFNTFRHVAACLMKYIPNMTSDR
AWELTNQVHYEGQAIVWVGPQEQAELYHEQLLRAGLTMAPLEPE;
(Sequence ID No. 7)
ASVVPQERQQDTRKHYPNYKVIVLNDDFNTFRHVAACLMKYIPNMTSDR
AWELTNQVHYKGQAIVWVGPQVQAELYHEQLLRAGLTMAPLEPG;
(Sequence ID No. 8)
ASVVPQERQQDTRKHYPNYKVIVLNDDFNTFRHVAACLMKYIPNMTSDR
AWELTNQVHYEGQAIVWVGPQVQAELYHEQLLRAGLTMAPLEPG;
(Sequence ID No. 9)
ASVVPQEGQQVTRKHYPNYKVIVLNDDFNTFQHVVACLKKYIPNMISDR
AWELTNQVHYGGRAIVWVGPQEQAELNHEQLLRAGLTMAPLEPE;
or
(Sequence ID No. 10)
ASVVPQERQQVARKHYPNYKVIVLNDDFNTFRHVAACLMKYIPNMTSDR
AWELTNQVHYEGQAIVWVGPQVQAELNHEQLLRAGLTMAPLEPE.

7. The process of embodiment 5, further comprising:
removing the N-terminal amino acid from the anchored analyte so that a penultimate residue becomes the N-terminal amino acid of the anchored analyte;
contacting the N-terminal amino acid of the anchored analyte with the binder complex;
selectively binding the amino acid-specific binder of the binder complex to the N-terminal amino acid of the anchored analyte when the N-terminal amino acid is the binding amino acid to form the tagged complex;
subjecting the taggant of the tagged complex to the stimulus;
producing, by the taggant of the tagged complex, the taggant signal in response to the stimulus;
detecting the taggant signal; and
identifying the N-terminal amino acid based on the taggant signal.
8. The process of embodiment 5, further comprising:
converting the N-terminal amino acid to an inert residue;
converting a penultimate residue to be the N-terminal amino acid when the inert residue is produced;
contacting the N-terminal amino acid of the anchored analyte with the binder complex;
selectively binding the amino acid-specific binder of the binder complex to the N-terminal amino acid of the anchored analyte when the N-terminal amino acid is the binding amino acid to form the tagged complex;
subjecting the taggant of the tagged complex to the stimulus;
producing, by the taggant of the tagged complex, the taggant signal in response to the stimulus;
detecting the taggant signal; and
identifying the N-terminal amino acid based on the taggant signal.
9. The process of embodiment 8, wherein converting the N-terminal amino acid to the inert residue comprises chemically changing the N-terminal amino acid prior to producing the inert residue.
10. The process of embodiment 9, wherein chemically changing the N-terminal amino acid prior to producing the inert residue comprises phosphorylating a free amine of the N-terminal amino acid.
11. A process for selectively isolating an analyte, the process comprising:
contacting an amino acid-specific binder with an analyte comprising a protein, a peptide, an amino acid, or a combination comprising at least one of foregoing;
selectively binding the amino acid-specific binder to the N-terminal amino acid of the analyte when the N-terminal amino acid comprises a binding amino acid to form an isolation complex;
separating the isolation complex from a fluid in which the isolation complex is disposed to selectively isolating the analyte,
wherein the amino acid-specific binder binds selectively to the binding amino acid, and
the amino acid-specific binder comprises an amino acid sequence comprising:
a first amino acid sequence comprising X1-GQQVT-X2-QHVV-X3-K-X4-MI-X5-GGR-X6-E-X7-Y-X8-E;
a second amino acid sequence comprising X1-RQQDT-X2-RHVA-X3-M-X4-MT-X5-EGQ-X6-E-X7-Y-X8-E;
a third amino acid sequence comprising X1-RQQVA-X2-RHVA-X3-M-X4-MT-X5-EGQ-X6-V-X7-Y-X8-G;
a fourth amino acid sequence comprising X1-RQQDT-X2-RHVA-X3-M-X4-KT-X5-EGQ-X6-E-X7-Y-X8-E;
a fifth amino acid sequence comprising X1-RQQVT-X2-RHVA-X3-M-X4-MT-X5-KGQ-X6-E-X7-N-X8-E;
a sixth amino acid sequence comprising X1-RQQVA-X2-RHVA-X3-M-X4-MT-X5-EGQ-X6-E-X7-Y-X8-E;
a seventh amino acid sequence comprising X1-RQQDT-X2-RHVA-X3-M-X4-MT-X5-KGQ-X6-V-X7-Y-X8-G; or
an eighth amino acid sequence comprising X1-RQQDT-X2-RHVA-X3-M-X4-MT-X5-EGQ-X6-V-X7-Y-X8-G;
wherein:
X1 comprises an amino acid sequence comprising ASVVPQE (Sequence ID No. 20);
X2 comprises an amino acid sequence comprising RKHYPNYKVIVLNDDFNTF (Sequence ID No. 21;
X3 comprises an amino acid sequence comprising ACL (Sequence ID No. 22);
X4 comprises an amino acid sequence comprising KYIPN (Sequence ID No. 23);
X5 comprises an amino acid sequence comprising SDRAWELTNQVHY (Sequence ID No. 24);
X6 comprises an amino acid sequence comprising AIVWVGPQ (Sequence ID No. 25);
X7 comprises an amino acid sequence comprising QAEL (Sequence ID No. 26); and
X8 comprises an amino acid sequence comprising HEQLLRAGLTMAPLEP (Sequence ID No. 27),
   such that a total percentage amount of substitutions and deletions to X1, X2, X3, X4, X5, X6, X7, and X8 is from 0% to less than 30%, exclusive of

(Sequence ID No. 28)
PSVVPQERQQVTRKHYPNYKVIVLNDDFNTFQHVAACLMKYIPNMTSDR
AWELTNQVHYEGQAIVWVGPQEQAELYHEQLLRAGLTMAPLEPE.

12. The process of embodiment 11, wherein the amino acid sequence comprises:

(Sequence ID No. 1)
ASVVPQEGQQVTRKHYPNYKVIVLNDDFNTFQHVVACLKKYIPNMISDR
AWELTNQVHYGGRAIVWVGPQEQAELYHEQLLRAGLTMAPLEPE;
(Sequence ID No. 2)
ASVVPQERQQDTRKHYPNYKVIVLNDDENTFRHVAACLMKYIPNMTSDR
AWELTNQVHYEGQAIVWVGPQEQAELYHEQLLRAGLTMAPLEPE;
(Sequence ID No. 3)
ASVVPQERQQVARKHYPNYKVIVLNDDFNTFRHVAACLMKYIPNMTSDR
AWELTNQVHYEGQAIVWVGPQVQAELYHEQLLRAGLTMAPLEPG;
(Sequence ID No. 4)
ASVVPQERQQDTRKHYPNYKVIVLNDDFNTFRHVAACLMKYIPNKTSDR
AWELTNQVHYEGQAIVWVGPQEQAELYHEQLLRAGLTMAPLEPE;
(Sequence ID No. 5)
ASVVPQERQQVTRKHYPNYKVIVLNDDENTFRHVAACLMKYIPNMTSDR
AWELTNQVHYKGQAIVWVGPQEQAELNHEQLLRAGLTMAPLEPE;
(Sequence ID No. 6)
ASVVPQERQQVARKHYPNYKVIVLNDDFNTFRHVAACLMKYIPNMTSDR
AWELTNQVHYEGQAIVWVGPQEQAELYHEQLLRAGLTMAPLEPE;
(Sequence ID No. 7)
ASVVPQERQQDTRKHYPNYKVIVLNDDFNTFRHVAACLMKYIPNMTSDR
AWELTNQVHYKGQAIVWVGPQVQAELYHEQLLRAGLTMAPLEPG;
(Sequence ID No. 8)
ASVVPQERQQDTRKHYPNYKVIVLNDDFNTFRHVAACLMKYIPNMTSDR
AWELTNQVHYEGQAIVWVGPQVQAELYHEQLLRAGLTMAPLEPG;
(Sequence ID No. 9)
ASVVPQEGQQVTRKHYPNYKVIVLNDDFNTFQHVVACLKKYIPNMISDR
AWELTNQVHYGGRAIVWVGPQEQAELNHEQLLRAGLTMAPLEPE;
or
(Sequence ID No. 10)
ASVVPQERQQVARKHYPNYKVIVLNDDFNTFRHVAACLMKYIPNMTSDR
AWELTNQVHYEGQAIVWVGPQVQAELNHEQLLRAGLTMAPLEPE.

13. The process of embodiment 11, wherein separating the isolation complex from the fluid in which the isolation complex is disposed comprises:
separating the isolation complex based on a size of the isolation complex relative to a size of other constituents in the fluid;
precipitating the isolation complex from the fluid;
centrifuging; or
a combination comprising at least one of the foregoing separations.
14. The process of embodiment 11, wherein the amino acid-specific binder is a member of a binder complex.
15. The process of embodiment 14, wherein the binder complex comprises:
the amino acid-specific binder; and
an adjunct attached to the amino acid-specific binder.
16. The process of embodiment 15, wherein the adjunct comprises a taggant, a protein, a substrate, or a chemical modifier.
17. The process of embodiment 16, wherein the taggant comprises:
a fluorescent moiety, an electrochemical moiety, or a combination comprising at least one of the foregoing moieties, and
the taggant produces a taggant signal in response to receiving a stimulus.
18. The process of embodiment 17, further comprising:
subjecting the taggant of the isolation complex to a stimulus;
producing, by the taggant of the isolation complex, a taggant signal in response to the stimulus;
detecting the taggant signal; and
identifying the N-terminal amino acid based on the taggant signal.
19. The process of embodiment 17, wherein the stimulus comprises a photon; and the taggant signal comprises fluorescence emitted from the taggant.
20. The process of embodiment 11, further comprising:
contacting the amino acid-specific binder with an adjunct to form a binder complex prior to contacting the amino acid-specific binder with the analyte.

What is claimed is:

1. A binder complex for selectively identifying an amino acid, the binder complex comprising:
an amino acid-specific binder; and
an adjunct attached to the amino acid-specific binder,
wherein the amino acid-specific binder binds selectively to a binding amino acid, and
the amino acid-specific binder comprises an amino acid sequence comprising:
a first amino acid sequence comprising X1-GQQVT-X2-QHVV-X3-K-X4-MI-X5-GGR-X6-E-X7-Y-X8-E;
a second amino acid sequence comprising X1-RQQDT-X2-RHVA-X3-M-X4-MT-X5-EGQ-X6-E-X7-Y-X8-E;
a third amino acid sequence comprising X1-RQQVA-X2-RHVA-X3-M-X4-MT-X5-EGQ-X6-V-X7-Y-X8-G;
a fourth amino acid sequence comprising X1-RQQDT-X2-RHVA-X3-M-X4-KT-X5-EGQ-X6-E-X7-Y-X8-E;
a fifth amino acid sequence comprising X1-RQQVT-X2-RHVA-X3-M-X4-MT-X5-KGQ-X6-E-X7-N-X8-E;
a sixth amino acid sequence comprising X1-RQQVA-X2-RHVA-X3-M-X4-MT-X5-EGQ-X6-E-X7-Y-X8-E;
a seventh amino acid sequence comprising X1-RQQDT-X2-RHVA-X3-M-X4-MT-X5-KGQ-X6-V-X7-Y-X8-G; or
an eighth amino acid sequence comprising X1-RQQDT-X2-RHVA-X3-M-X4-MT-X5-EGQ-X6-V-X7-Y-X8-G;
wherein;
X1 comprises an amino acid sequence comprising ASVVPQE (Sequence ID No. 20);
X2 comprises an amino acid sequence comprising
(Sequence ID No. 21)
RKHYPNYKVIVLNDDFNTF;
X3 comprises an amino acid sequence comprising ACL (Sequence ID No. 22);
X4 comprises an amino acid sequence comprising KYIPN (Sequence ID No. 23);
X5 comprises an amino acid sequence comprising SDRAWELTNQVHY (Sequence ID No. 24);
X6 comprises an amino acid sequence comprising AIVWVGPQ (Sequence ID No. 25);
X7 comprises an amino acid sequence comprising QAEL (Sequence ID No. 26); and
X8 comprises an amino acid sequence comprising HEQLLRAGLTMAPLEP (Sequence ID No. 27),
such that a total percentage amount of substitutions and deletions to X1, X2, X3, X4, X5, X6, X7, and X8 is from 0% to less than 30%, exclusive of
(Sequence ID No. 28)
PSVVPQERQQVTRKHYPNYKVIVLNDDENTFQHVAACLMKYIPNMTSDR
AWELTNQVHYEGQAIVWVGPQEQAELYHEQLLRAGLTMAPLEPE.

2. The binder complex of claim 1, wherein the amino acid sequence comprises:
(Sequence ID No. 1)
ASVVPQEGQQVTRKHYPNYKVIVLNDDFNTFQHVVACLKKYIPNMISDR
AWELTNQVHYGGRAIVWVGPQEQAELYHEQLLRAGLTMAPLEPE;
(Sequence ID No. 2)
ASVVPQERQQDTRKHYPNYKVIVLNDDENTFRHVAACLMKYIPNMTSDR
AWELTNQVHYEGQAIVWVGPQEQAELYHEQLLRAGLTMAPLEPE;
(Sequence ID No. 3)
ASVVPQERQQVARKHYPNYKVIVLNDDFNTFRHVAACLMKYIPNMTSDR
AWELTNQVHYEGQAIVWVGPQVQAELYHEQLLRAGLTMAPLEPG;
(Sequence ID No. 4)
ASVVPQERQQDTRKHYPNYKVIVLNDDFNTFRHVAACLMKYIPNKTSDR
AWELTNQVHYEGQAIVWVGPQEQAELYHEQLLRAGLTMAPLEPE;
(Sequence ID No. 5)
ASVVPQERQQVTRKHYPNYKVIVLNDDENTFRHVAACLMKYIPNMTSDR
AWELTNQVHYKGQAIVWVGPQEQAELNHEQLLRAGLTMAPLEPE;
(Sequence ID No. 6)
ASVVPQERQQVARKHYPNYKVIVLNDDFNTFRHVAACLMKYIPNMTSDR
AWELTNQVHYEGQAIVWVGPQEQAELYHEQLLRAGLTMAPLEPE;
(Sequence ID No. 7)
ASVVPQERQQDTRKHYPNYKVIVLNDDFNTFRHVAACLMKYIPNMTSDR
AWELTNQVHYKGQAIVWVGPQVQAELYHEQLLRAGLTMAPLEPG;
(Sequence ID No. 8)
ASVVPQERQQDTRKHYPNYKVIVLNDDFNTFRHVAACLMKYIPNMTSDR
AWELTNQVHYEGQAIVWVGPQVQAELYHEQLLRAGLTMAPLEPG;
(Sequence ID No. 9)
ASVVPQEGQQVTRKHYPNYKVIVLNDDFNTFQHVVACLKKYIPNMISDR
AWELTNQVHYGGRAIVWVGPQEQAELNHEQLLRAGLTMAPLEPE;
or
(Sequence ID No. 10)
ASVVPQERQQVARKHYPNYKVIVLNDDFNTFRHVAACLMKYIPNMTSDR
AWELTNQVHYEGQAIVWVGPQVQAELNHEQLLRAGLTMAPLEPE.

3. The binder complex of claim 1, wherein the adjunct comprises a taggant, a protein, a substrate, or a chemical modifier.

4. The binder complex of claim 3, wherein the taggant comprises:
a fluorescent moiety, an electrochemical moiety, or a combination comprising at least one of the foregoing moieties, and the taggant produces a taggant signal in response to receiving a stimulus.

5. A process for selectively identifying an N-terminal amino acid, the process comprising:
providing an analyte comprising a protein, a peptide, an amino acid, or a combination comprising at least one of foregoing;
contacting a C-terminal end of the analyte with an anchor;
anchoring the C-terminal end to the anchor to form an anchored analyte;
contacting an N-terminal amino acid of the anchored analyte with a binder complex, the binder complex comprising:
an amino acid-specific binder; and
a taggant attached to the amino acid-specific binder;
selectively binding the amino acid-specific binder of the binder complex to the N-terminal amino acid of the anchored analyte when the N-terminal amino acid comprises a binding amino acid to form a tagged complex;
subjecting the taggant of the tagged complex to a stimulus;
producing, by the taggant of the tagged complex, a taggant signal in response to the stimulus;
detecting the taggant signal; and
identifying the N-terminal amino acid based on the taggant signal,
wherein the amino acid-specific binder binds selectively to the binding amino acid, and
the amino acid-specific binder comprises an amino acid sequence comprising:
a first amino acid sequence comprising X1-GQQVT-X2-QHVV-X3-K-X4-MI-X5-GGR-X6-E-X7-Y-X8-E;
a second amino acid sequence comprising X1-RQQDT-X2-RHVA-X3-M-X4-MT-X5-EGQ-X6-E-X7-Y-X8-E;
a third amino acid sequence comprising X1-RQQVA-X2-RHVA-X3-M-X4-MT-X5-EGQ-X6-V-X7-Y-X8-G;
a fourth amino acid sequence comprising X1-RQQDT-X2-RHVA-X3-M-X4-KT-X5-EGQ-X6-E-X7-Y-X8-E;
a fifth amino acid sequence comprising X1-RQQVT-X2-RHVA-X3-M-X4-MT-X5-KGQ-X6-E-X7-N-X8-E;
a sixth amino acid sequence comprising X1-RQQVA-X2-RHVA-X3-M-X4-MT-X5-EGQ-X6-E-X7-Y-X8-E;
a seventh amino acid sequence comprising X1-RQQDT-X2-RHVA-X3-M-X4-MT-X5-KGQ-X6-V-X7-Y-X8-G; or
an eighth amino acid sequence comprising X1-RQQDT-X2-RHVA-X3-M-X4-MT-X5-EGQ-X6-V-X7-Y-X8-G;
wherein:
X1 comprises an amino acid sequence comprising ASVVPQE (Sequence ID No. 20);
X2 comprises an amino acid sequence comprising RKHYPNYKVIVLNDDFNTF (Sequence ID No. 21);
X3 comprises an amino acid sequence comprising ACL (Sequence ID No. 22);
X4 comprises an amino acid sequence comprising KYIPN (Sequence ID No. 23);
X5 comprises an amino acid sequence comprising SDRAWELTNQVHY (Sequence ID No. 24);
X6 comprises an amino acid sequence comprising AIVWVGPQ (Sequence ID No. 25);
X7 comprises an amino acid sequence comprising QAEL (Sequence ID No. 26); and
X8 comprises an amino acid sequence comprising HEQLLRAGLTMAPLEP (Sequence ID No. 27),
such that a total percentage amount of substitutions and deletions to X1, X2, X3, X4, X5, X6, X7, and X8 is from 0% to less than 30%, exclusive of
(Sequence ID No. 28)
PSVVPQERQQVTRKHYPNYKVIVLNDDFNTFQHVAACLMKYIPNMTSDR
AWELTNQVHYEGQAIVWVGPQEQAELYHEQLLRAGLTMAPLEPE.

6. The process of claim 5, wherein the amino acid sequence comprises:
(Sequence ID No. 1)
ASVVPQEGQQVTRKHYPNYKVIVLNDDFNTFQHVVACLKKYIPNMISDR
AWELTNQVHYGGRAIVWVGPQEQAELYHEQLLRAGLTMAPLEPE;
(Sequence ID No. 2)
ASVVPQERQQDTRKHYPNYKVIVLNDDENTFRHVAACLMKYIPNMTSDR
AWELTNQVHYEGQAIVWVGPQEQAELYHEQLLRAGLTMAPLEPE;
(Sequence ID No. 3)
ASVVPQERQQVARKHYPNYKVIVLNDDFNTFRHVAACLMKYIPNMTSDR
AWELTNQVHYEGQAIVWVGPQVQAELYHEQLLRAGLTMAPLEPG;
(Sequence ID No. 4)
ASVVPQERQQDTRKHYPNYKVIVLNDDFNTFRHVAACLMKYIPNKTSDR
AWELTNQVHYEGQAIVWVGPQEQAELYHEQLLRAGLTMAPLEPE;
(Sequence ID No. 5)
ASVVPQERQQVTRKHYPNYKVIVLNDDENTFRHVAACLMKYIPNMTSDR
AWELTNQVHYKGQAIVWVGPQEQAELNHEQLLRAGLTMAPLEPE;
(Sequence ID No. 6)
ASVVPQERQQVARKHYPNYKVIVLNDDFNTFRHVAACLMKYIPNMTSDR
AWELTNQVHYEGQAIVWVGPQEQAELYHEQLLRAGLTMAPLEPE;
(Sequence ID No. 7)
ASVVPQERQQDTRKHYPNYKVIVLNDDFNTFRHVAACLMKYIPNMTSDR
AWELTNQVHYKGQAIVWVGPQVQAELYHEQLLRAGLTMAPLEPG;
(Sequence ID No. 8)
ASVVPQERQQDTRKHYPNYKVIVLNDDFNTFRHVAACLMKYIPNMTSDR
AWELTNQVHYEGQAIVWVGPQVQAELYHEQLLRAGLTMAPLEPG;
(Sequence ID No. 9)
ASVVPQEGQQVTRKHYPNYKVIVLNDDFNTFQHVVACLKKYIPNMISDR
AWELTNQVHYGGRAIVWVGPQEQAELNHEQLLRAGLTMAPLEPE;
or
(Sequence ID No. 10)
ASVVPQERQQVARKHYPNYKVIVLNDDFNTFRHVAACLMKYIPNMTSDR
AWELTNQVHYEGQAIVWVGPQVQAELNHEQLLRAGLTMAPLEPE.

7. The process of claim 5, further comprising:
removing the N-terminal amino acid from the anchored analyte so that a penultimate residue becomes the N-terminal amino acid of the anchored analyte;
contacting the N-terminal amino acid of the anchored analyte with the binder complex;
selectively binding the amino acid-specific binder of the binder complex to the N-terminal amino acid of the anchored analyte when the N-terminal amino acid is the binding amino acid to form the tagged complex;
subjecting the taggant of the tagged complex to the stimulus;
producing, by the taggant of the tagged complex, the taggant signal in response to the stimulus;
detecting the taggant signal; and
identifying the N-terminal amino acid based on the taggant signal.

8. The process of claim 5, further comprising:
converting the N-terminal amino acid to an inert residue;
converting a penultimate residue to be the N-terminal amino acid when the inert residue is produced;
contacting the N-terminal amino acid of the anchored analyte with the binder complex;
selectively binding the amino acid-specific binder of the binder complex to the N-terminal amino acid of the anchored analyte when the N-terminal amino acid is the binding amino acid to form the tagged complex;
subjecting the taggant of the tagged complex to the stimulus;
producing, by the taggant of the tagged complex, the taggant signal in response to the stimulus;
detecting the taggant signal; and
identifying the N-terminal amino acid based on the taggant signal.

9. The process of claim 8, wherein converting the N-terminal amino acid to the inert residue comprises chemically changing the N-terminal amino acid prior to producing the inert residue.

10. The process of claim 9, wherein chemically changing the N-terminal amino acid prior to producing the inert residue comprises phosphorylating a free amine of the N-terminal amino acid.

11. A process for selectively isolating an analyte, the process comprising:
contacting an amino acid-specific binder with an analyte comprising a protein, a peptide, an amino acid, or a combination comprising at least one of foregoing;
selectively binding the amino acid-specific binder to the N-terminal amino acid of the analyte when the N-terminal amino acid comprises a binding amino acid to form an isolation complex;
separating the isolation complex from a fluid in which the isolation complex is disposed to selectively isolating the analyte,
wherein the amino acid-specific binder binds selectively to the binding amino acid, and
the amino acid-specific binder comprises an amino acid sequence comprising:
a first amino acid sequence comprising X1-GQQVT-X2-QHVV-X3-K-X4-MI-X5-GGR-X6-E-X7-Y-X8-E;
a second amino acid sequence comprising X1-RQQDT-X2-RHVA-X3-M-X4-MT-X5-EGQ-X6-E-X7-Y-X8-E;
a third amino acid sequence comprising X1-RQQVA-X2-RHVA-X3-M-X4-MT-X5-EGQ-X6-V-X7-Y-X8-G;
a fourth amino acid sequence comprising X1-RQQDT-X2-RHVA-X3-M-X4-KT-X5-EGQ-X6-E-X7-Y-X8-E;
a fifth amino acid sequence comprising X1-RQQVT-X2-RHVA-X3-M-X4-MT-X5-KGQ-X6-E-X7-N-X8-E;
a sixth amino acid sequence comprising X1-RQQVA-X2-RHVA-X3-M-X4-MT-X5-EGQ-X6-E-X7-Y-X8-E;
a seventh amino acid sequence comprising X1-RQQDT-X2-RHVA-X3-M-X4-MT-X5-KGQ-X6-V-X7-Y-X8-G; or
an eighth amino acid sequence comprising X1-RQQDT-X2-RHVA-X3-M-X4-MT-X5-EGQ-X6-V-X7-Y-X8-G;
wherein:
X1 comprises an amino acid sequence comprising ASVVPQE (Sequence ID No. 20);
X2 comprises an amino acid sequence comprising RKHYPNYKVIVLNDDFNTF (Sequence ID No. 21);
X3 comprises an amino acid sequence comprising ACL (Sequence ID No. 22);
X4 comprises an amino acid sequence comprising KYIPN (Sequence ID No. 23);
X5 comprises an amino acid sequence comprising SDRAWELTNQVHY (Sequence ID No. 24);
X6 comprises an amino acid sequence comprising AIVWVGPQ (Sequence ID No. 25);
X7 comprises an amino acid sequence comprising QAEL (Sequence ID No. 26); and
X8 comprises an amino acid sequence comprising HEQLLRAGLTMAPLEP (Sequence ID No. 27),
such that a total percentage amount of substitutions and deletions to X1, X2, X3, X4, X5, X6, X7, and X8 is from 0% to less than 30%, exclusive of
(Sequence ID No. 28)
PSVVPQERQQVTRKHYPNYKVIVLNDDFNTFQHVAACLMKYIPNMTSDR
AWELTNQVHYEGQAIVWVGPQEQAELYHEQLLRAGLTMAPLEPE.

12. The process of claim 11, wherein the amino acid sequence comprises:
(Sequence ID No. 1)
ASVVPQEGQQVTRKHYPNYKVIVLNDDFNTFQHVVACLKKYIPNMISDR
AWELTNQVHYGGRAIVWVGPQEQAELYHEQLLRAGLTMAPLEPE;
(Sequence ID No. 2)
ASVVPQERQQDTRKHYPNYKVIVLNDDENTFRHVAACLMKYIPNMTSDR
AWELTNQVHYEGQAIVWVGPQEQAELYHEQLLRAGLTMAPLEPE;
(Sequence ID No. 3)
ASVVPQERQQVARKHYPNYKVIVLNDDFNTFRHVAACLMKYIPNMTSDR
AWELTNQVHYEGQAIVWVGPQVQAELYHEQLLRAGLTMAPLEPG;
(Sequence ID No. 4)
ASVVPQERQQDTRKHYPNYKVIVLNDDFNTFRHVAACLMKYIPNKTSDR
AWELTNQVHYEGQAIVWVGPQEQAELYHEQLLRAGLTMAPLEPE;
(Sequence ID No. 5)
ASVVPQERQQVTRKHYPNYKVIVLNDDENTFRHVAACLMKYIPNMTSDR
AWELTNQVHYKGQAIVWVGPQEQAELNHEQLLRAGLTMAPLEPE;
(Sequence ID No. 6)
ASVVPQERQQVARKHYPNYKVIVLNDDFNTFRHVAACLMKYIPNMTSDR
AWELTNQVHYEGQAIVWVGPQEQAELYHEQLLRAGLTMAPLEPE;
(Sequence ID No. 7)
ASVVPQERQQDTRKHYPNYKVIVLNDDFNTFRHVAACLMKYIPNMTSDR
AWELTNQVHYKGQAIVWVGPQVQAELYHEQLLRAGLTMAPLEPG;
(Sequence ID No. 8)
ASVVPQERQQDTRKHYPNYKVIVLNDDFNTFRHVAACLMKYIPNMTSDR
AWELTNQVHYEGQAIVWVGPQVQAELYHEQLLRAGLTMAPLEPG;
(Sequence ID No. 9)
ASVVPQEGQQVTRKHYPNYKVIVLNDDFNTFQHVVACLKKYIPNMISDR
AWELTNQVHYGGRAIVWVGPQEQAELNHEQLLRAGLTMAPLEPE;
or
(Sequence ID No. 10)
ASVVPQERQQVARKHYPNYKVIVLNDDFNTFRHVAACLMKYIPNMTSDR
AWELTNQVHYEGQAIVWVGPQVQAELNHEQLLRAGLTMAPLEPE.

13. The process of claim 11, wherein separating the isolation complex from the fluid in which the isolation complex is disposed comprises:
separating the isolation complex based on a size of the isolation complex relative to a size of other constituents in the fluid;
precipitating the isolation complex from the fluid;
centrifuging; or
a combination comprising at least one of the foregoing separations.

14. The process of claim 11, wherein the amino acid-specific binder is a member of a binder complex.

15. The process of claim 14, wherein the binder complex comprises:
the amino acid-specific binder; and
an adjunct attached to the amino acid-specific binder.

16. The process of claim 15, wherein the adjunct comprises a taggant, a protein, a substrate, or a chemical modifier.

17. The process of claim 16, wherein the taggant comprises:
a fluorescent moiety, an electrochemical moiety, or a combination comprising at least one of the foregoing moieties, and
the taggant produces a taggant signal in response to receiving a stimulus.

18. The process of claim 17, further comprising:
subjecting the taggant of the isolation complex to a stimulus;
producing, by the taggant of the isolation complex, a taggant signal in response to the stimulus;
detecting the taggant signal; and
identifying the N-terminal amino acid based on the taggant signal.

19. The process of claim 17, wherein the stimulus comprises a photon; and
the taggant signal comprises fluorescence emitted from the taggant.

20. The process of claim 11, further comprising:
contacting the amino acid-specific binder with an adjunct to form a binder complex prior to contacting the amino acid-specific binder with the analyte.
